(19) United States
(12) Patent Application Publication
(10) Pub. No.: US 2010/0016173 A1
(43) Pub. Date: Jan. 21, 2010

(54) MATERNAL SERUM BIOMARKERS FOR DETECTION OF PRE-ECLAMPSIA

(75) Inventors: SRINIVASA R. NAGALLA, HILLSBORO, OR (US); JUHA RASANEN, OULU (FI); MICHAEL GRAVETT, SEATTLE, WA (US)

Correspondence Address: Goodwin Procter LLP;Attn: Patent Administrator, 135 Commonwealth Drive, Menlo Park, CA, 94025-1105, US

(73) Assignee: PROTEOGENIX, INC., COSTA MESA, CA (US)

(21) Appl. No.: 12/363,659

(22) Filed: Jan. 30, 2009

Related U.S. Application Data
(60) Provisional application No. 61/024,859, filed on Jan. 30, 2008.

(51) Int. Cl. G01N 33/53; C12Q 1/02; C40B 30/04

(52) U.S. Cl. 506/9; 435/29; 435/7.1

(57) ABSTRACT

The present invention concerns the identification and detection of maternal serum biomarkers of pre-eclampsia and associated complications, gestational hypertension and placental insufficiency using global proteomic approaches. The invention further concerns the identification of maternal serum biomarkers for detection of pre-eclampsia and associated complications, gestational hypertension and placental insufficiency during early gestation.

### RELATED APPLICATION

This application claims priority under 35 U.S.C. § 119(e) to U.S. provisional application No. 61/024,859, filed Jan. 30, 2008, the entire contents of which are incorporated herein by reference.

### BACKGROUND OF THE INVENTION

1. Field of the Invention

The present invention concerns the identification and detection of maternal serum biomarkers of pre-eclampsia using global proteomic approaches. The invention further concerns the identification of maternal serum biomarkers for detection of pre-eclampsia during early gestation.

2. Description of the Related Art

Preeclampsia, a transient disorder unique to pregnancy, affects 5% to 10% of pregnant women (Solomon, 2006). It is a major cause of maternal morbidity and mortality worldwide, and is also associated with a five-fold increase in perinatal mortality (Solomon, 2006; Roberts, 2003). Importantly, it is unpredictable in onset and disease progression, and is cured only by delivery.

Preeclampsia is defined as new onset hypertension and proteinuria after 20 weeks gestation in a previously normotensive pregnant woman and can be mild or severe. Patients with mild disease have blood pressures >140/90 and proteinuria with >300 mg protein noted on a 24 hour urine noted after 20 weeks gestation and usually deliver near term without significant co-morbidities. However, about 25% of preeclampsia is severe, characterized by symptoms of central nervous system dysfunction, hepatocellular injury, reduced urine output, and markedly elevated blood pressure (systolic>160 mmHg or diastolic>110 mmHg). Severe preeclampsia occurs frequently in the late second and early third trimester, and is associated with marked increases in both maternal and perinatal morbidity and mortality. Two severe complications of preeclampsia are 1) HELLP syndrome characterized by hemolysis, elevated liver enzymes, and low platelets and 2) eclampsia—characterized by the development of seizures. Both of these conditions are rare occurrences but are associated with poor prognosis (Solomon, 2006)

There are multiple risk factors associated with preeclampsia.[2, 3] These include nulliparity, history of preeclampsia in prior pregnancy, extremes in age (<18 years and >40 years), family history of preeclampsia, chronic hypertension, chronic renal disease, antiphospholipid antibody syndrome or inherited thrombophilia, vascular or connective tissue disease, diabetes mellitus, multiple gestation, obesity, male partner whose previous partner had preeclampsia, hydrops fetalis and unexplained fetal intrauterine growth restriction. However, preeclampsia is primarily a disorder of otherwise healthy young women during their first pregnancy. More than 50% of cases occur among these otherwise young, low risk, nulliparous patients

Unfortunately, the pathophysiology of preeclampsia is unclear and the diagnosis based entirely upon clinical criteria (Roberts, 2003). Recent data suggests that events leading to preeclampsia may begin as silently as early as the first trimester. Unfortunately, there are no clinically useful screening tests to predict the development of preeclampsia (Conde-Agudelo, 2004). Recent reports suggest that an imbalance of vasoactive placental peptides may be useful in the early prediction of preeclampsia.

These peptides include soluble fms-like tyrosine kinase-1 (sFlt-1) (Maynard, 2003), endoglin (Levine, 2006), placental growth factor and vascular endothelial growth factor (Polliotti 2003). Soluble fms-like tyrosine kinase-1 (sFlt-1) and endoglin are both antiangiogenic peptides and are produced in excess 2-3 months prior to development of preeclampsia (Maynard, 2003; Levine, 2006). In contrast, placental growth factor and vascular endothelial growth factor promote angiogenesis. Both have been shown to be significantly lower in second trimester maternal sera of women who subsequently develop severe pre-eclampsia (Polliotti 2003). Taken together, these observations suggest that preeclampsia is preceded by abnormal placentation and early invasion of maternal uterine spiral arteries that begins in the first trimester. More recently, we have utilized proteomic analysis to characterize 9 peptides, including acute phase reactants and vasoactive peptides, differentially expressed in the first trimester among women who subsequently develop preeclampsia when compared to women who do not (Rasanen, 2006). Ultimately these vascular perturbations lead to oxidative damage to the endothelium of small arterioles, leading to hypertension and multi-organ dysfunction. Early recognition of these peptides in the first trimester, before extensive endothelial damage may allow for early intervention and prevention trials.

The only cure for preeclampsia is delivery of the baby and placenta. Disease progression follows no predictable pattern; therefore, beyond 37 weeks of gestation (term), delivery is warranted. At gestational ages of less than 34 weeks, treatment of hypertension, and close fetal surveillance may prevent cerebral vascular accidents and prolong the pregnancy, but do not treat the underlying disease process. Delivery is still warranted for development of severe preeclampsia or eclampsia (Sibai, 2007). During labor, women with preeclampsia are at risk for development of eclampsia. The MAGPIE study demonstrated that administration of magnesium sulphate to women with pre-eclampsia reduces the risk of an eclamptic seizure (Altman, 2002). This risk is reduced from 4-7% to less than 1% with the use of IV magnesium sulfate. Magnesium sulfate is typically bolused with 4 grams IV followed by a continuous infusion of 2 grams per hour throughout labor and 24 hours postpartum (44% of eclampsia occurs postpartum) to reduce the risk of seizures.

Since the only treatment for preeclampsia is delivery, screening and prevention strategies prior to the onset of disease would be beneficial. Unfortunately, there is no preventative therapy for preeclampsia (Sibai, 2007). Because the pathophysiology ultimately leads to oxidative endothelial damage and microvascular coagulopathy, studies have utilized a variety of antioxidant therapy (Chappell, 1999; Rumbold 2005; Rumbold, 2006), antiplatelet therapy (Duley, 2007), or calcium supplementation (Vilar, 2006) to reduce the risks of preeclampsia among high risk women. Unfortunately, these treatments, generally begun in the second trimester, have resulted in either no, or only very modest reduction, in subsequent development of preeclampsia. This may be attributable to delayed screening of high risk women (because of inadequate screening tests) or delayed treatment for a process that began weeks earlier, in the first trimester. This points to the need for reliable screening test in the first trimester that will allow earlier, and potentially more efficacious, treatment and prevention strategies. Reductions in the risk of preeclampsia and its associated morbidities may well depend upon earlier identification of patients at risk.

### SUMMARY OF THE INVENTION

In one aspect, the invention provides a method for the diagnosis of active pre-eclampsia and associated complications in a pregnant female mammalian subject comprising testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia. In certain embodiments, the complications include small for gestational age and/or HELLP syndrome.

In one embodiment, the subject is a human patient.

In some embodiments, the methods comprising testing the abundance of at least three, at least four or all of said proteins, in any combination.

In one embodiment, the methods comprise testing the level of proteins fibronectin (Q8IVI8), choriogonadotropin subunit beta (P1233), matrix metalloproteinase-9 (P14780) and pappalysin-2 (Q9BXP8), and diagnosing said subject with pre-eclampsia, if two or more of said tested proteins shows a statistically significant difference in the maternal serum sample relative to normal maternal serum. In certain embodiments, the diagnosis of a subject with preeclampsia is made if all of said tested proteins show a statistically significant difference in the maternal serum sample relative to normal maternal serum.

In certain embodiments, level is determined by an immunoassay, by mass spectrometry, and/or by using a protein array.

In yet another aspect, the invention provides an immunoassay kit comprising antibodies and reagents for the detection of one or more proteins selected from the group consisting of alpha-1B-glycoprotein (P04217), actin (P62736), apolipoprotein B-100 (Q13787), apolipoprotein C-II (P02655), apolipoprotein C-III (P02656), C4b-binding protein beta chain (P20851), cathepsin D (P07339), choriogonadotropin subunit beta (P1233), cholinesterase (P06276), chorionic somatomammotropin hormone (P01243), cystatin-C (P01034), endoglin (P17813), coagulation factor XI (P03951), coagulation factor VII (P08709), fibronectin (Q8IVI8), filamin-A (P21333), heparin cofactor 2 (P05546), hepatocyte growth factor-like protein (P26927), histidine-rich glycoprotein (P04196), insulin-like growth factor-binding protein 2 (P18065), laminin subunit beta-1 (P07942), lipopolysaccharide-binding protein (P18428), matrix metalloproteinase-9 (P14780), pappalysin-2 (Q9BXP8), plastin-2 (P13796), profiling-1 (P07737), pregnancy-specific bet-1-glycoprotein (P11464), receptor-type tyrosine-protein phosphatase gamma (P23470), pregnancy zone protein (P20742), plasma retinol-binding protein (P02753), SH3 domain-binding glutamic acid-rich-like protein 3 (Q9H299), trangelin-2 (P37802), talin-1 (Q9Y490), tropomyosin alpha-4-chain (P67936), vasorin (Q6EMK4), vascular endothelial growth factor receptor 3 (P35916), vinculin (P18206), von Willebrand factor (P04275).

In one aspect, the invention provides an immunoassay kit comprising antibodies and reagents for the detection of one or more proteins selected from the group consisting of cystatin-C (P01034), endoglin (P17813), fibronectin (Q8IVI8), apolipoprotein C-III (P02656), choriogonadotropin subunit beta (P1233) and pappalysin-2 (Q9BXP8). In one embodiment, the kit includes antibodies and reagents for the detection of all of said proteins.

In another aspect, the invention provides an immunoassay kit comprising antibodies and reagents for the detection of one or more proteins selected from the group consisting of cystatin-C (P01034), endoglin (P17813), fibronectin (Q8IVI8), apolipoprotein C-III (P02656), and pappalysin-2 (Q9BXP8). In one embodiment, the kit includes antibodies and reagents for the detection of all of said proteins.

In yet another aspect, the invention provides an immunoassay kit comprising antibodies and reagents for the detection of one or more proteins selected from the group consisting of fibronectin (Q8IVI8), pappalysin-2 (Q9BXP8), and matrix metalloproteinase-9 (P14780). In one embodiment, the kit includes antibodies and reagents for the detection of all of said proteins.

In still another aspect, the invention provides a report comprising the results of and/or diagnosis based on a test comprising testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia.

In yet another aspect, the invention provides a tangible medium storing the results of and/or diagnosis based on a test comprising testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia.

In one other aspect, the invention provides a method for the diagnosis of pre-eclampsia in a female mammalian subject in early gestation comprising: testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and diagnosing said subject with pre-eclampsia if said level shows a statistically significant difference relative to the level in said normal maternal serum, or does not show a statistically significant difference relative to the level in said maternal serum known to be indicative of pre-eclampsia.

In one embodiment, the subject is a human patient.

In certain embodiments, the subject is about 9 to about 11 weeks gestation. In other embodiments, the subject is about 10 to about 14 weeks gestation. In one embodiment, the pre-eclampsia is severe pre-eclampsia.

In other embodiments, the methods include testing the level of at least three, at least four, at least five, at least six, etc. of the listed proteins, in any combination.

In certain embodiments, the methods include testing the level of proteins complement factor D (P00746), vascular cell adhesion protein-1 (P19320), pappalysin-1 (Q13219), endoglin (P17813), plasma retinol-binding protein (P02753), and choriogonadotropin subunit beta (P01233). In certain other embodiments, the methods include testing the level of proteins membrane copper amine oxidase (Q16853), C-reactive protein (P02741), Serum amyloid P-component (P02743), catalase, tubulin beta, plasma retinol binding protein, lipopolysaccharide binding protein, and chorionic somatomammotropin. In yet other embodiments, the methods include testing the level of proteins pappalysin-1 (SEQ ID NO: 63), vascular cell adhesion protein 1 (SEQ ID NO: 60), beta-2-microglobulin (SEQ ID NO: 45), and cystatin C (SEQ ID NO: 11). In still other embodiments, the methods include testing the level of proteins C-reactive protein (P02741), vascular cell adhesion protein-1 (P19320), pappalysin-1 (Q13219), beta-2-microglobulin (P61769), and plasma retinol-binding protein (P02753).

In certain embodiments, level is determined by an immunoassay, by mass spectrometry, and/or by using a protein array.

In one aspect, the invention further includes an immunoassay kit comprising antibodies and reagents for the detection of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040).

In another aspect, the invention includes an immunoassay kit comprising antibodies and reagents for the detection of two or more proteins selected from the group consisting of complement factor D (P00746), vascular cell adhesion protein-1 (P19320), and pappalysin-1 (Q13219). In one embodiment, the kit includes antibodies and reagents for the detection of all of said proteins.

In yet another aspect, the invention provides an immunoassay kit comprising antibodies and reagents for the detection of two or more proteins selected from the group consisting of complement factor D (P00746), vascular cell adhesion protein-1 (P19320), pappalysin-1 (Q13219), endoglin (P17813), choriogonadoropin subunit beta (P01233) and plasma retinol-binding protein (P02753). In one embodiment, the kit includes antibodies and reagents for the detection of all of said proteins.

In still another aspect, the invention provides an immunoassay kit comprising antibodies and reagents for the detection of two or more proteins selected from the group consisting of pappalysin-1 (Q13219), C-reactive protein (P02741), plasma retinol-binding protein (P02753), beta-2-microglobulin (P61769) and vascular cell adhesion protein 1 (P19320). In one embodiment, the kit includes antibodies and reagents for the detection of all of said proteins.

In another aspect, the invention provides a report comprising the results of and/or diagnosis based on a test comprising testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and diagnosing said subject with pre-eclampsia if said level shows a statistically significant difference relative to the level in said normal maternal serum, or does not show a statistically significant difference relative to the level in said maternal serum known to be indicative of pre-eclampsia.

In still another aspect, the invention provides a tangible medium storing the results of and/or diagnosis based on a test comprising testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and diagnosing said subject with pre-eclampsia if said level shows a statistically significant difference relative to the level in said normal maternal serum, or does not show a statistically significant difference relative to the level in said maternal serum known to be indicative of pre-eclampsia.

In one aspect, the invention further provides a method for the diagnosis of gestational hypertension in a pregnant female mammalian subject comprising: testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO: 115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and diagnosing said subject with gestational hypertension if said level shows a statistically significant difference relative to the level in said normal maternal serum, or does not show a statistically significant difference relative to the level in said maternal serum known to be indicative of gestational hypertension.

In a specific embodiment, the subject is a human patient.

In other embodiments, the methods include testing the level of at least three, at least four, at least five, at least six, etc. of the listed proteins, in any combination.

In one embodiment, the methods include testing the level of proteins Pappalysin-2 (SEQ ID NO: 38), choriogonadotropin subunit beta (SEQ ID NO: 8), histidine rich glycoprotein (SEQ ID NO: 19), plasma retinol-binding protein (SEQ ID NO: 29), Matrix metalloproteinase-9 (SEQ ID NO: 23), Apolipoprotein B-100 (SEQ ID NO: 3), endoglin (SEQ ID NO: 12), and Vascular endothelial growth factor receptor 1 (SEQ ID NO: 121).

In certain embodiments, level is determined by an immunoassay, by mass spectrometry, and/or by using a protein array.

In another aspect, the invention provides a report comprising the results of and/or diagnosis based on a test comprising testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO: 115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and diagnosing said subject with gestational hypertension if said level shows a statistically significant difference relative to the level in said normal maternal serum, or does not show a statistically significant difference relative to the level in said maternal serum known to be indicative of gestational hypertension.

In another aspect, the invention provides a tangible medium storing the results of and/or diagnosis based on a test comprising testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO: 115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and diagnosing said subject with gestational hypertension if said level shows a statistically significant difference relative to the level in said normal maternal serum, or does not show a statistically significant difference relative to the level in said maternal serum known to be indicative of gestational hypertension.

In one other aspect, the invention provides a method for the diagnosis of placental insufficiency in a pregnant female mammalian subject having preeclampsia comprising testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and diagnosing said subject with placental insufficiency if said level shows a statistically significant difference relative to the level in said normal maternal serum, or does not show a statistically significant difference relative to the level in said maternal serum known to be indicative of placental insufficiency.

In a specific embodiment, the subject is a human patient.

In other embodiments, the methods include testing the level of at least three, at least four, etc. of the listed proteins, in any combination.

In certain embodiments, level is determined by an immunoassay, by mass spectrometry, and/or by using a protein array.

In another aspect, the invention provides a report comprising the results of and/or diagnosis based on a test comprising testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and diagnosing said subject with placental insufficiency if said level shows a statistically significant difference relative to the level in said normal maternal serum, or does not show a statistically significant difference relative to the level in said maternal serum known to be indicative of placental insufficiency.

In still another aspect, the invention provides a tangible medium storing the results of and/or diagnosis based on a test comprising testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and diagnosing said subject with placental insufficiency if said level shows a statistically significant difference relative to the level in said normal maternal serum, or does not show a statistically significant difference relative to the level in said maternal serum known to be indicative of placental insufficiency.

In one embodiment, the methods of the invention include the testing is implemented using an apparatus adapted to determine the level of said proteins. In another embodiment, the testing is performed by using a software program executed by a suitable processor. In certain embodiments, the program is embodied in software stored on a tangible medium. In certain other embodiments, the tangible medium is selected from the group consisting of a flash drive, a CD-ROM, a floppy disk, a hard drive, a DVD, and a memory associated with the processor.

In certain embodiments, the methods of the invention further comprise the step of preparing a report recording the results of said testing or the diagnosis. In one embodiment, the report is recorded or stored on a tangible medium. In another embodiment, the tangible medium is paper. In other embodiments, the tangible medium is selected from the group consisting of a flash drive, a CD-ROM, a floppy disk, a hard drive, a DVD, and a memory associated with the processor.

In one embodiment, the methods of the invention further comprise the step of communicating the results of said diagnosis to an interested party. In certain embodiments, the interested party is the patient or the attending physician. In certain other embodiments, the communication is in writing, by email, or by telephone.

In another aspect, the invention concerns the use of proteins in the preparation or manufacture of proteomic profiles as a means for the early determination of the state of a maternal or fetal condition, e.g., preeclampsia, gestational hypertension, and/or placental insufficiency.

### BRIEF DESCRIPTION OF THE DRAWINGS

FIG. 1 depicts the performance of combinations of candidate protein biomarkers for classifying samples with or without pre-eclampsia (sensitivity at FP=20% indicated).

### DETAILED DESCRIPTION OF THE PREFERRED EMBODIMENT

### I. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

The term “proteome” is used herein to describe a significant portion of proteins in a biological sample at a given time. The concept of proteome is fundamentally different from the genome. While the genome is virtually static, the proteome continually changes in response to internal and external events.

The term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Thus the proteomic profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel. Differential expression profiles may have important diagnostic value, even in the absence of specifically identified proteins. Single protein spots can then be detected, for example, by immunoblotting, multiple spots or proteins using protein microarrays. The proteomic profile typically represents or contains information that could range from a few peaks to a complex profile representing 50 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50, or at least 60, or at least 65, or at least 70, or at least 75, or at least 80, or at least 85, or at least 85, or at least 90, or at least 95, or at least 100, or at least 125, or at least 150, or at least 175, or at least 200 proteins.

The term “biological fluid” as used herein refers to refers to liquid material derived from a human or other animal. Biological fluids include, but are not limited to, cord blood, neonatal serum, cerebrospinal fluid (CSF), cervical-vaginal fluid (CVF), amniotic fluid, serum, plasma, urine, cerebrospinal fluid, breast milk, mucus, saliva, and sweat.

By “pre-eclampsia” is meant the multi-system disorder that is characterized by hypertension with proteinuria or edema, or both, glomerular dysfunction, brain edema, liver edema, or coagulation abnormalities due to pregnancy or the influence of a recent pregnancy and all complications associated with the disorder. Pre-eclampsia generally occurs after the 20^{th} week of gestation. Pre-eclampsia is generally defined as some combination of the following symptoms: (1) a systolic blood pressure (BP)>140 mmHg and a diastolic BP>90 mmHg after 20 weeks gestation (generally measured on two occasions, 4-168 hours apart), (2) new onset proteinuria (1+ by dipstick on urinanaysis, >300 mg of protein in a 24-hour urine collection, or a single random urine sample having a protein/creatinine ratio>0.3), and (3) resolution of hypertension and proteinuria by 12 weeks postpartum. Severe pre-eclampsia is generally defined as (1) a diastolic BP>110 mmHg (generally measured on two occasions, 4-168 hours apart) or (2) proteinuria characterized by a measurement of 3.5 g or more protein in a 24-hour urine collection or two random urine specimens with at least 3+ protein by dipstick. In pre-eclampsia, hypertension and proteinuria generally occur within seven days of each other. In severe pre-eclampsia, severe hypertension, severe proteinuria and HELLP syndrome (hemolysis, elevated liver enzymes, low platelets) or eclampsia can occur simultaneously or only one symptom at a time. Occasionally, severe pre-eclampsia can lead to the development of seizures. This severe form of the syndrome is referred to as “eclampsia.” Eclampsia can also include dysfunction or damage to several organs or tissues such as the liver (e.g., hepatocellular damage, periportal necrosis) and the central nervous system (e.g., cerebral edema and cerebral hemorrhage). The etiology of the seizures is thought to be secondary to the development of cerebral edema and focal spasm of small blood vessels in the kidney. Preeclampsia is associated with fetal complications such as intrauterine growth retardation (IUGR) and small for gestational age (SGA).

By “small for gestational age (SGA)” is meant a fetus whose birth weight is a weight less than 2,500 gm (5 lbs. 8 oz.) or below the 10^{th} percentile for gestational age according to U.S. tables of birth weight for gestational age by race, parity, and infant sex as defined by World Health Organization (WHO) (Zhang and Bowes, Obstet. Gynecol. 86:200-208, 1995).

“Patient response” can be assessed using any endpoint indicating a benefit to the patient, including, without limitation, (1) inhibition, at least to some extent, of the progression of a pathologic condition, (2) prevention of the pathologic condition, (3) relief, at least to some extent, of one or more symptoms associated with the pathologic condition; (4) increase in the length of survival following treatment; and/or (5) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The designation of any particular protein, as used herein, includes all fragments, precursors, and naturally occurring variants, such as alternatively spliced and allelic variants and isoforms, as well as soluble forms of the protein named, along with native sequence homologs (including all naturally occurring variants) in other species. Thus, for example, when it is stated that the abundance of haptoglobin precursor (Swiss-Prot Acc. No. P00738) is tested, the statement specifically includes testing any fragments, precursers, or naturally occurring variant of the protein listed under Swiss-Prot Acc. No. P00738, as well as its non-human homologs and naturally occurring variants thereof, if subject is non-human.

### II. Detailed Description

The present invention concerns, in one aspect, methods and means for an early, reliable and non-invasive testing of pre-eclampsia and associated complications in pregnant women by proteomic analysis of maternal serum. The invention further concerns, in another aspect, identification of biomarkers of pre-eclampsia, including pre-eclampsia during early gestation, such as in the first trimester of pregnancy, e.g., during 9 to 11 weeks, and also during 10 to 14 weeks, using proteomics techniques. In another aspect, the invention concerns methods and means for an early, reliable and non-invasive testing of gestational hypertension, or pregnancy-induced hypertension, in pregnant women by proteomic analysis of maternal serum. In yet another aspect, methods and means for an early, reliable and non-invasive testing of placental insufficiency in pregnant women by proteomic analysis of maternal serum. In another aspect, the invention concerns the use of proteins in the preparation or manufacture of proteomic profiles as a means for the early determination of the state of a maternal or fetal condition, e.g., preeclampsia, gestational hypertension, and/or placental insufficiency. The invention utilizes proteomics techniques well known in the art, as described, for example, in the following textbooks, the contents of which are hereby expressly incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

One skilled in the art will recognize many methods and materials similar or equivalent to those described herein, which could be used in the practice of the present invention. Indeed, the present invention is in no way limited to the methods and materials described.

1. Identification of Proteins and Polypeptides Expressed in Biological Fluids

According to the present invention, proteomics analysis of biological fluids can be performed using a variety of methods known in the art. Biological fluids include, for example, cervical-vaginal fluid (CVF), amniotic fluid, serum, plasma, urine, cerebrospinal fluid, breast milk, mucus, and saliva.

Typically, protein patterns (proteome maps) of samples from different sources, such as normal biological fluid (normal sample) and a test biological fluid (test sample), are compared to detect proteins that are up- or down-regulated in a disease. These proteins can then be excised for identification and full characterization, e.g. using peptide-mass fingerprinting and/or mass spectrometry and sequencing methods, or the normal and/or disease-specific proteome map can be used directly for the diagnosis of the disease of interest, or to confirm the presence or absence of the disease.

In comparative analysis, it is important to treat the normal and test samples exactly the same way, in order to correctly represent the relative abundance of proteins, and obtain accurate results. The required amount of total proteins will depend on the analytical technique used, and can be readily determined by one skilled in the art. The proteins present in the biological samples are typically separated by two-dimensional gel electrophoresis (2-DE) according to their pI and molecular weight. The proteins are first separated by their charge using isoelectric focusing (one-dimensional gel electrophoresis). This step can, for example, be carried out using immobilized pH-gradient (IPG) strips, which are commercially available. The second dimension is a normal SDS-PAGE analysis, where the focused IPG strip is used as the sample. After 2-DE separation, proteins can be visualized with conventional dyes, like Coomassie Blue or silver staining, and imaged using known techniques and equipment, such as, e.g. Bio-Rad GS800 densitometer and PDQUEST software, both of which are commercially available. Individual spots are then cut from the gel, destained, and subjected to tryptic digestion. The peptide mixtures can be analyzed by mass spectrometry (MS). Alternatively, the peptides can be separated, for example by capillary high pressure liquid chromatography (HPLC) and can be analyzed by MS either individually, or in pools.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionisation (ESI) methods. There are several versions of mass analyzer, including, for example, MALDI-TOF and triple or quadrupole-TOF, or ion trap mass analyzer coupled to ESI. Thus, for example, a Q-Tof-2 mass spectrometer utilizes an orthogonal time-of-flight analyzer that allows the simultaneous detection of ions across the full mass spectrum range. For further details see, e.g. Chemusevich et al., J. Mass Spectrom. 36:849-865 (2001).

If desired, the amino acid sequences of the peptide fragments and eventually the proteins from which they derived can be determined by techniques known in the art, such as certain variations of mass spectrometry, or Edman degradation.

2. Early Detection of Pre-Eclampsia and Related Complications

Preeclampsia, defined as maternal hypertension accompanied by proteinuria, edema, or both, occurs in 7% of pregnancies not terminating in the first trimester. Although the cause is unknown, it is more common in extremes of age in childbearing, maternal diabetes, pregnancies with multiple gestations, and pre-existing maternal renal disease and or hypertension. Preeclampsia is associated with increases in perinatal mortality, and may also lead to eclampsia, characterized by maternal seizures and increased maternal mortality.

Complications of preeclampsia include intrauterine growth retardation (IUGR), small for gestational age (SGA) and HELLP syndrome. Small for Gestational Age (SGA) babies are those whose birth weight lies below the 10^{th} percentile for that gestational age (see above). The incidence of SGA in developed countries is 8.1%. Pre-eclampsia is a condition known to be associated with intrauterine fetal growth restriction (IUGR) and SGA. The etiology, however, can be maternal, fetal or placental. Fetal risk factors include, for example, chromosomal abnormality and infection. Maternal risk factors include, for example, preeclampsia, thrombophilias, antiphospholipid syndrome, defective placentation, sickle cell anemia, drug use, alcohol, and smoking. Accurate diagnosis is complicated by ultra sound assessments and accurate estimation of gestational age. Development of early and reliable markers for SGA is imperative to allow for therapy and intervention to optimize the outcome for the neonate and mother.

HELLP, a syndrome consisting of Hemolysis, Elevated liver enzyme Levels and Low Platelet count, is an obstetric complication that is frequently misdiagnosed at initial presentation. HELLP syndrome occurs in approximately 0.2 to 0.6 percent of all pregnancies. The mainstay of therapy is supportive management, including seizure prophylaxis and blood pressure control in patients with hypertension. Because the symptoms of HELLP syndrome are variable, diagnosis is often delayed. Early diagnosis, however, is critical, and thus, development of early and reliable markers for HELLP syndrome is imperative to allow for therapy and intervention to optimize the outcome for the neonate and mother.

Currently the mainstay of therapy for preeclampsia is delivery and anticonvulsant prophylaxis with magnesium sulfate. Prior to the advent of magnesium sulfate therapy, the observed maternal mortality was 20-30%. However, with prompt diagnosis, allowing anticonvulsant therapy with magnesium sulfate, anti-hypertensives, and delivery the maternal mortality has been reduced to near zero.

Unfortunately, the diagnosis of preeclampsia based upon commonly recognized symptoms and signs is frequently difficult, and occurs late in the course of the disease. Frequently fetal compromise in growth or well-being is the first recognized manifestation of preeclampsia. Laboratory markers for preeclampsia include quantitation of proteinuria, and elevated serum concentrations of uric acid or creatinine. There are no currently available serum markers for early preeclampsia or markers which identify women which will develop preeclampsia. Recently prospective serum markers including leptin and uric acid have been associated with subsequent preeclampsia in one study (Gursoy T, et al. Preeclampsia disrupts the normal physiology of leptin.: Am J Perinatol.19(6):303-10, 2002) but much work is needed to confirm these findings. Development of early and reliable markers for preeclampsia and related complications is imperative to allow for therapy and intervention to minimize associated complications and optimize the outcome for the neonate and mother.

3. Early Detection and Diagnosis of Pre-Eclampsia Using Maternal Serum Biomarkers

The present invention provides reliable, non-invasive method for the diagnosis of the pre-eclampsia using biomarkers identified in the maternal serum using a proteomics approach. The diagnosis can be performed any time during pregnancy, including early gestation, including the first trimester. In one embodiment, the diagnosis can be performed between about 9 and about 11 gestational weeks. In another embodiment, the diagnosis can be performed between about 10 and about 14 weeks.

As noted before, in the context of the present invention the term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Although it is possible to identify and sequence all or some of the proteins present in the proteome of a biological fluid, this is not necessary for the diagnostic use of the proteomic profiles generated in accordance with the present invention. Diagnosis of a particular disease can be based on characteristic differences (unique expression signatures) between a normal proteomic profile, and proteomic profile of the same biological fluid obtained under the same circumstances, when the disease or pathologic condition to be diagnosed is present. The unique expression signature can be any unique feature or motif within the proteomic profile of a test or reference biological sample that differs from the proteomic profile of a corresponding normal biological sample obtained from the same type of source, in a statistically significant manner. For example, if the proteomic profile is presented in the form of a mass spectrum, the unique expression signature is typically a peak or a combination of peaks that differ, qualitatively or quantitatively, from the mass spectrum of a corresponding normal sample. Thus, the appearance of a new peak or a combination of new peaks in the mass spectrum, or any statistically significant change in the amplitude or shape of an existing peak or combination of existing peaks, or the disappearance of an existing peak, in the mass spectrum can be considered a unique expression signature. When the proteomic profile of the test sample obtained from a mammalian subject is compared with the proteomic profile of a reference sample comprising a unique expression signature characteristic of a pathologic maternal or fetal condition, the mammalian subject is diagnosed with such pathologic condition if it shares the unique expression signature with the reference sample.

A particular pathologic maternal/fetal condition can be diagnosed by comparing the proteomic profile of a biological fluid obtained from the subject to be diagnosed with the proteomic profile of a normal biological fluid of the same kind, obtained and treated the same manner. If the proteomic profile of the test sample is essentially the same as the proteomic profile of the normal sample, the subject is considered to be free of the subject pathologic maternal/fetal condition. If the proteomic profile of the test sample shows a unique expression signature relative to the proteomic profile of the normal sample, the subject is diagnosed with the maternal/fetal condition in question.

Alternatively or in addition, the proteomic profile of the test sample may be compared with the proteomic profile of a reference sample, obtained from a biological fluid of a subject independently diagnosed with the pathologic maternal/fetal condition ion question. In this case, the subject is diagnosed with the pathologic condition if the proteomic profile of the test sample shares at least one feature, or a combination of features representing a unique expression signature, with the proteomic profile of the reference sample.

Statistical methods for comparing proteomic profiles are well known in the art. For example, in the case of a mass spectrum, the proteomic profile is defined by the peak amplitude values at key mass/charge (M/Z) positions along the horizontal axis of the spectrum. Accordingly, a characteristic proteomic profile can, for example, be characterized by the pattern formed by the combination of spectral amplitudes at given M/Z vales. The presence or absence of a characteristic expression signature, or the substantial identity of two profiles can be determined by matching the proteomic profile (pattern) of a test sample with the proteomic profile (pattern) of a reference or normal sample, with an appropriate algorithm. A statistical method for analyzing proteomic patterns is disclosed, for example, in Petricoin III, et al., The Lancet 359:572-77 (2002).; Issaq et al., Biochem Biophys Commun 292:587-92 (2002); Ball et al., Bioinformatics 18:395-404 (2002); and Li et al., Clinical Chemistry Journal, 48:1296-1304 (2002).

In a particular embodiment, the diagnostic tests of the present invention are performed in the form of protein arrays or immunoassays.

4. Maternal Serum Biomarkers of Gestational Hypertension Distinct From Preeclampsia

Gestational (transient) hypertension, or pregnancy-induced hypertension, is generally characterized as the acute onset of hypertension (systolic blood pressure≧140, diastolic blood pressure≧90, measured at least 6 hours apart on two occasions) in pregnancy or the early puerperium without proteinuria or abnormal edema and resolving within 10 days after delivery. As treatment options differ for gestational hypertension and preeclampsia, there is a need for reliable diagnosis of gestational hypertension that could distinguish from preeclampsia and thus facilitate early intervention strategies.

Thus, in one aspect, the present invention provides reliable, non-invasive methods for the diagnosis of gestational hypertension, or pregnancy-induced hypertension, distinct from preeclampsia. In one embodiment, the present invention provides a multi-analyte panel of serum biomarkers for gestational hypertension.

5. Maternal Serum Biomarkers of Placental Insufficiency in Preeclampsia

Preeclampsia (PE) and fetal growth restriction are associated with placental insufficiency, which is defined as insufficient blood flow to the placenta during pregnancy. The early prediction of placental insufficiency associated with PE may lead to novel, early interventions to prevent fetal growth restriction. Thus, in one aspect, the present invention provides reliable, non-invasive methods for the diagnosis of placental insufficiency using biomarkers identified in the maternal serum using a proteomics approach. The diagnosis can be performed any time during pregnancy, including early gestation, including the first trimester. In one embodiment, the diagnosis can be performed at about 9 to about 11 gestational weeks. In another embodiment, the diagnosis can be performed at about 10 to about 14 weeks.

6. Protein Arrays

In recent years, protein arrays have gained wide recognition as a powerful means to detect proteins, monitor their expression levels, and investigate protein interactions and functions. They enable high-throughput protein analysis, when large numbers of determinations can be performed simultaneously, using automated means. In the microarray or chip format, that was originally developed for DNA arrays, such determinations can be carried out with minimum use of materials while generating large amounts of data.

Although proteome analysis by 2D gel electrophoresis and mass spectrometry, as described above, is very effective, it does not always provide the needed high sensitivity and this might miss many proteins that are expressed at low abundance. Protein microarrays, in addition to their high efficiency, provide improved sensitivity.

Protein arrays are formed by immobilizing proteins on a solid surface, such as glass, silicon, micro-wells, nitrocellulose, PVDF membranes, and microbeads, using a variety of covalent and non-covalent attachment chemistries well known in the art. The solid support should be chemically stable before and after the coupling procedure, allow good spot morphology, display minimal nonspecific binding, should not contribute a background in detection systems, and should be compatible with different detection systems.

In general, protein microarrays use the same detection methods commonly used for the reading of DNA arrays. Similarly, the same instrumentation as used for reading DNA microarrays is applicable to protein arrays.

Thus, capture arrays (e.g. antibody arrays) can be probed with fluorescently labelled proteins from two different sources, such as normal and diseased biological fluids. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy.

For further details, see, for example, Zhou H, et al., Trends Biotechnol. 19:S34-9 (2001); Zhu et al., Current Opin. Chem. Biol. 5:40-45-(2001); Wilson and Nock, Angew Chem Int Ed Engl 42:494-500 (2003); and Schweitzer and Kingsmore, Curr Opin Biotechnol 13:14-9 (2002). Biomolecule arrays are also disclosed in U.S. Pat. No. 6,406,921, issued Jun. 18, 2002, the entire disclosure of which is hereby expressly incorporated by reference.

7. Immunoassays

The diagnostic assays of the present invention can also be performed in the form of various immunoassay formats, which are well known in the art. There are two main types of immunoassays, homogenous and heterogenous. In homogenous immunoassays, both the immunological reaction between an antigen and an antibody and the detection are carried out in a homogenous reaction. Heterogeous immunoassays include at least one separation step, which allows the differentiation of reaction products from unreacted reagents.

ELISA is a heterogenous immunoassay, which has been widely used in laboratory practice since the early 1970's. The assay can be used to detect antigensin various formats.

In the “sandwich” format the antigen being assayed is held between two different antibodies. In this method, a solid surface is first coated with a solid phase antibody. The test sample, containing the antigen (i.e. a diagnostic protein), or a composition containing the antigen, being measured, is then added and the antigen is allowed to react with the bound antibody. Any unbound antigen is washed away. A known amount of enzyme-labelled antibody is then allowed to react with the bound antigen. Any excess unbound enzyme-linked antibody is washed away after the reaction. The substrate for the enzyme used in the assay is then added and the reaction between the substrate and the enzyme produces a colour change. The amount of visual colour change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labelled antigen and both compete for binding to an anti-antigen antibody attached to a solid surface. Excess free enzyme-labelled antigen is washed off before the substrate for the enzyme is added. The amount of color intensity resulting from the enzyme-substrate interaction is a measure of the amount of antigen in the sample tested.

Homogenous immunoassays include, for example, the Enzyme Multiplied Immunoassay Technique (EMIT), which typically includes a biological sample comprising the compound or compounds to be measured, enzyme-labeled molecules of the compound(s) to be measured, specific antibody or antibodies binding the compound(s) to be measured, and a specific enzyme chromogenic substrate. In a typical EMIT excess of specific antibodies is added to a biological sample. If the biological sample contains the proteins to be detected, such proteins bind to the antibodies. A measured amount of the corresponding enzyme-labelled proteins is then added to the mixture. Antibody binding sites not occupied by molecules of the protein in the sample are occupied with molecules of the added enzyme-labelled protein. As a result, enzyme activity is reduced because only free enzyme-labelled protein can act on the substrate. The amount of substrate converted from a colourless to a coloured form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the Ag-enzyme complex is Ab-bound makes the EMIT a unique system, enabling the test to be performed without a separation of bound from unbound compounds as is necessary with other immunoassay methods.

Part of this invention is also an immunoassay kit. In one aspect, the invention includes a sandwich immunoassay kit comprising a capture antibody and a detector antibody. The capture antibody and detector antibody can be monoclonal or polyclonal. In another aspect, the invention includes a diagnostic kit comprising lateral flow devices, such as immunochromatographic strip (ICS) tests, using immunoflowchromatography. The lateral flow devices employ lateral flow assay techniques as generally described in U.S. Pat. Nos. 4,943,522; 4,861,711; 4,857,453; 4,855,240; 4,775,636; 4,703,017; 4,361, 537; 4,235,601; 4,168,146; 4,094,647, the entire contents of each of which is incorporated by reference. In yet another aspect, the immunoassay kit may comprise, for example, in separate containers (a) monoclonal antibodies having binding specificity for the polypeptides used in the diagnosis of a particular maternal/fetal condition, such as preeclampsia; (b) and anti-antibody immunoglobulins. This immunoassay kit may be utilized for the practice of the various methods provided herein. The monoclonal antibodies and the anti-antibody immunoglobulins may be provided in an amount of about 0.001 mg to about 100 grams, and more preferably about 0.01 mg to about 1 gram. The anti-antibody immunoglobulin may be a polyclonal immunoglobulin, protein A or protein G or functional fragments thereof, which may be labeled prior to use by methods known in the art. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like. The test kit may be packaged in any suitable manner, typically with all elements in a single container along with a sheet of printed instructions for carrying out the test.

8. Diagnostic and Treatment Methods

The diagnostic methods of the present invention are valuable tools for practicing physicians to make quick treatment decisions, which are often critical for the survival of the infant and/or mother. Thus, for example, if a pregnant woman shows symptoms of pre-ecplampsia, gestational hypertension or placental insufficiency, it is important to take immediate steps to treat the condition and improve the chances of the survival of the fetus and limit the risks to the mother's health.

Following the measurement or obtainment of the expression levels of the proteins identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the expression level in a test subject of one or more of the biomarkers herein is communicated to the subject as soon as possible after the assay is completed and the diagnosis and/or prediction is generated. The one or more biomarkers identified and quantified in the methods described herein can be contained in one or more panels. The number of biomarkers comprising a panel can include 1 biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, 20 biomarkers, etc. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

To facilitate diagnosis, the reference and/or subject biomarker profiles or expression level of one or more of the biomarkers presented herein of the present invention can be displayed on a display device, contained electronically, or in a machine-readable medium, such as but not limited to, analog tapes like those readable by a VCR, CD-ROM, DVD-ROM, USB flash media, e.g., flash drive, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

Further details of the invention will be apparent from the following non-limiting examples. All references cited throughout the disclosure, and the references cited therein, are expressly incorporated by reference herein.

### Example 1

### Identification of Maternal Serum Biomarkers of Pre-Eclampsia Using Global Proteomic Approaches

Experimental Methods

Sample Collection and Processing (Active PE): A total of 118 human subjects (control n=58, mild PE n=30 and severe PE n=30) were identified prospectively and given informed consent to participate in the study. The mean gestational age of the women at the collection are 33.94±4.31 (control), 35.0±5.58 (mild PE) and 31.24±6.27 weeks (severe PE). All the samples were allowed to clot for 30 min., spun down at 5000 g, supernatant was collected and stored in −80° C. until further processing. Pre-eclampsia was defined as (ACOG criteria) systolic blood pressure of >140 mmHg or diastolic blood pressure>90 mmHg on at least two occasions, 4 hours to 1 week apart and protenuria (>300 mg in a 24 hour urine collection or 2+ on dip stick measurement). Severe pre-eclampsia is defined as systolic blood pressure of >160 mmHg, diastolic blood pressure>110 mmHg and/or protenuria (>300 mg or 3+ on dip stick measurement). All the samples were allowed to clot for 30 min., spun down at 3000 g, supernatant was collected and stored at −80° C. until further processing.

Multidimensional Liquid Chromatography Tandem Mass Spectrometry (LC-LC-MS/MS; MudPIT): A total of 1 mg each of individually pooled control, mild and severe preeclampsia serum samples (8 samples/pool) were digested with trypsin, separated into 95 fractions using SCX chromatography and analyzed on a Q-toF-2 mass spectrometer connected to a CapLC (Waters, Inc., Milford, Mass.). Data were searched against a Swiss-Prot human database (version 46.6) as perscribed in previous publication (Gravett, MG. IAI). Spectral counting, the total number of MS/MS spectra matched to a particular protein, has been used to assess the relative abundance of a protein in a sample. (Pang, Ginanni et al. 2002; Zybailov, Mosley et al. 2006; Nagalla, Canick et al. 2007)

Enzyme-Linked Immunosorbent Assay: Concentrations of biomarker proteins in control, mild and severe preeclampsia serum samples were estimated by enzyme-linked immunosorbent assay (ELISA) (Clark and Adams 1977; Nerurkar, Namba et al. 1984).

Specific antibodies and pure proteins for Apolipoprotein B-100 (ApoB), Cystatin-C (CystatinC), Endoglin (Endoglin), Fibronectin (Fibronectin), Plasma retinol-binding protein (RBP), Apolipoprotein C-III (ApoCIII), Chorionic somatomammotropin hormone (CSH1), Choriogonadotropin subunit beta (βHCG), Pappalysin-2 (PAPPA2), Vascular endothelial growth factor receptor 3 (VEGFR3), Histidine-rich glycoprotein (HPRG), Insulin-like growth factor-binding protein 2 (IGFBP2), Matrix metalloproteinase-9 (MMP9), pregnancy-specific-β-1-glycoprotein 1 (PSG1), were obtained either from Dako, RND or Academy biomed. For the sandwich ELISA, a capture antibody and a detection antibody were used. To facilitate the detection, the antibodies were conjugated with either biotin or horse radish peroxidase (HRP) using Sulfo-NHS-Biotinylation kit (Pierce Biotechnology Inc., Rockford, Ill.). Pure proteins were used as the standards in the assay.

ELISA plates were prepared by coating with an appropriate capture antibody, in 0.1 M carbonate bicarbonate buffer, pH 9.6, at 4° C. over night. Appropriate dilutions of the standard proteins and serum samples were prepared in 1% BSA, and incubated in the pre-coated plates in triplicate, at a volume of 100 μL/well. A reference serum sample was also assayed in every plate for calculating the plate-to-plate variation. All the incubation steps were done at room temperature for 1 hr. After each incubation steps, the wells were washed with PBST using a power washer (Tecan). Followed by the antigen binding, a biotinylated detection antibody was incubated. Horseradish peroxidase (HRP) conjugated streptavidin was used as the detection components, and tetramethyl benizidine (TMB) reagent (Neogen Corporation) was used as the substrate to develop the color. The reaction was finally stopped by adding 100 μL of 2NH₂SO₄, and the optical density (OD) was measured at 450 nm.

A standard curve was generated for every ELISA plate by plotting concentrations of the known proteins samples against their OD values, using Softmax Pro (Molecular Devices Corporation). The concentrations of the individual proteins were estimated from the average values of triplicates in comparison to the standard curve. Since the samples were processed in multiple plates, a reference standard (known concentration of pure proteins) was spotted on all the plates and the ELISA values from all the plates are normalized with respect to that standard in order to correct for plate-to-plate variation, and then transformed to natural log scale.

Statistical Analysis of MudPIT data: Maternal serum proteins with at least three unique peptide identifications in at least one sample are considered for label-free quantitation (spectral counting). In order to reduce false positive rate, protein entries were further curated before subjecting to spectral counting. Shared spectral counts of non-degenerate proteins that belong to same family and have significant sequence homology (>50%) were combined into single entry. Shared spectral counts of non-degenerate proteins that did not fit afore-mentioned criteria were assigned to one of the protein using Occam's razor approach. Spectral counts of all Immunoglobulin and pregnancy-specific-β-1-glycoprotein variants are collapsed into single entries. Curated proteins were then subjected to independent pair-wise comparisons to determine differentially expressed proteins between control and PE.

Pair-wise comparison was performed using either a 2×2 chi-square test or fisher exact test. Normalization of spectral counts to account for experimental variability was built into the pair-wise comparisons. The method was automated using a SAS program (version 9.1) and all proteins were independently tested. Level of significance was set at 0.05, The fold expression change of differentially expressed proteins was quantified using the equation described as previously published (Old, Meyer-Arendt et al. 2005).

Statistical Analysis of ELISA data: Candidate protein biomarker concentrations (expressed as ng/mL) measured by ELISA experiments in cohorts of active PE (control (n=58), mild preeclampsia (n=30), and severe preeclampsia (n=30) and first trimester screening (control (n=96), mild preeclampsia (n=33), and severe preeclampsia (n=40). maternal serum samples were log transformed before subjecting them to statistical analysis. Subjects with adequate overall protein in their samples, but with ELISA values under detectable limit for a particular protein were assigned a value of 0.1 rather than 0 to facilitate log-transformation. When transformed to log scale, the value of −2.3 corresponded to those without any protein detected. Independent pair-wise comparisons of log-transformed protein concentrations between control and preeclampsias were performed using one-way analysis of variance (ANOVA) test. The average value on the log-scale values was transformed back to original units (harmonic mean) for presentation. The comparisons of the control group to the latter two groups with preeclampsia were performed as well and receiver-operator characteristic (ROC) curves were constructed to examine the predictive potential of selected biomarkers, singly and in combination. The Bonferroni correction was applied to adjust for multiple comparisons.

To explore the possibility that two or more markers might be combined to improve classification accuracy, the multi-variable logistic regression models were fit to develop risk scores (predicted probabilities obtained from models). Based on results from single proteins, the classification performance of several different combinations of 2, 3 or 4 proteins were evaluated. ROC curves, and other corresponding measures, were computed based on each of the multi-protein models to choose the most promising combination.

The descriptive and comparative analyses, logistic regression models, and ROC curves were conducted using SAS software (v9.1).

Results

The demographic statistics are presented below in Table 1. Serum draws of 8 subjects from control, mild PE and severe PE were subjected to two-dimensional liquid chromatography tandem mass spectrometry (2-DLC/MS/MS). Selected protein biomarkers from 2-DLC/MS/MS are validated using Enzyme-Linked Immunosorbent Assays.

TABLE 1
Demographic Statistics.

| | | | | | Std | | |
|---|---|---|---|---|---|---|---|
| condition | N | Variable | N | Mean | Dev | Min | Max |
| control | 58 | Gestion age at | 58 | 33.94 | 4.31 | 22.00 | 39.00 |
| | | collection | | | | | |
| Gestational | | Gestion age at | 14 | 34.75 | 4.71 | 23.70 | 38.00 |
| Hypertension | | collection | 14 | 34.53 | 4.52 | 20.00 | 39.00 |
| (PIH) | 14 | Gestion age at | | | | | |
| | | PE start | | | | | |
| Severe PE | | Gestion age at | 30 | 31.24 | 6.27 | 10.30 | 37.70 |
| | | collection | | | | | |
| | 30 | Gestion age at | 30 | 31.17 | 4.00 | 21.00 | 36.00 |
| | | PE start | | | | | |
| Mild PE | | Gestion age at | 30 | 35.00 | 5.58 | 8.10 | 39.40 |
| | | collection | | | | | |
| | 30 | Gestion age at | 30 | 35.51 | 2.54 | 31.00 | 40.00 |
| | | PE start | | | | | |

A total of 457 unique proteins were identified in this study 38 differentially abundant maternal serum proteins are summarized in Table 2 below. Independent pair-wise differences in abundance between samples were performed for each protein from women with and without PE. Proteins with a relative expression change of ≧1.5 fold and passes the chi-square test with a p-value≦0.05 in any of the comparisons are considered as potentially differentially expressed between the samples. These included pregnancy proteins such as Choriogonadotropin subunit beta and Pappalysin-2, and extracellular matrix signaling factors such as Fibronectin and Matrix metalloproteinase-9.

TABLE 2
Serum Proteins Sharing Significant Changes between Pair-wise Comparisons of
Control, mild PE and severe PE Samples

| | | Mild PE | | vs. Control | |
|---|---|---|---|---|---|
| | | vs. Control | | Severe PE | |
| Swiss-Prot | | Fold | | Fold | |
| Accession | Description | Change^{a} | p-value | Change^{a} | p-value |
| P04217 | Alpha-1B-glycoprotein (SEQ ID NO: 1) | 1.0 | 0.63 | 1.6 | <0.0001 |
| P62736^{b} | Actin (SEQ ID NO: 2) | −1.1 | 0.29 | −2.8 | <0.0001 |
| Q13787^{c} | Apolipoprotein B-100 (SEQ ID NO: 3) | −1.1 | <0.0001 | −3.8 | <0.0001 |
| P02655 | Apolipoprotein C-II (SEQ ID NO: 4) | 1.1 | 0.77 | 4.7 | <0.0001 |
| P02656^{c} | Apolipoprotein C-III (SEQ ID NO: 5) | 2.1 | <0.0001 | 2.9 | <0.0001 |
| P20851 | C4b-binding protein beta chain (SEQ ID NO: 6) | −1.1 | 0.81 | 2 | 0.014 |
| P07339 | Cathepsin D (SEQ ID NO: 7) | 4.9 | 0.063 | 6.8 | 0.0083 |
| P01233^{c} | Choriogonadotropin subunit beta (SEQ ID NO: 8) | −7.6 | 0.0036 | 2.2 | 0.03 |
| P06276 | Cholinesterase (SEQ ID NO: 9) | 1.1 | 0.79 | 2.1 | 0.0044 |
| P01243^{c} | Chorionic somatomammotropin hormone (SEQ ID | −2.9 | 0.0002 | −2.2 | 0.0029 |
| | NO: 10) | | | | |
| P01034^{c} | Cystatin-C (SEQ ID NO: 11) | 6.5 | 0.016 | 10.4 | 0.0003 |
| P17813^{c} | Endoglin (SEQ ID NO: 12) | −1.0 | | 10.4 | 0.0003 |
| P03951 | Coagulation factor XI (SEQ ID NO: 13) | −1.1 | 0.82 | 1.9 | 0.017 |
| P08709 | Coagulation factor VII (SEQ ID NO: 14) | −1.2 | 0.75 | 2.4 | 0.023 |
| Q8IVI8^{c} | Fibronectin (SEQ ID NO: 15) | 2.1 | <0.0001 | 10.9 | <0.0001 |
| P21333 | Filamin-A (SEQ ID NO: 16) | 1.0 | 0.87 | −30 | <0.0001 |
| P05546 | Heparin cofactor 2 (SEQ ID NO: 17) | −1.1 | 0.37 | −1.8 | <0.0001 |
| P26927 | Hepatocyte growth factor-like protein (SEQ ID | 1.3 | 0.34 | 2.2 | 0.0005 |
| | NO: 18) | | | | |
| P04196^{c} | Histidine-rich glycoprotein (SEQ ID NO: 19) | 1.3 | 0.0036 | 1.7 | <0.0001 |
| P18065^{c} | Insulin-like growth factor-binding protein 2 (SEQ ID | −2.7 | | 2.9 | 0.068 |
| | NO: 20) | | | | |
| P07942 | Laminin subunit beta-1 (SEQ ID NO: 21) | 2.5 | | 9.7 | 0.0006 |
| P18428 | Lipopolysaccharide-binding protein (SEQ ID NO: 22) | −1.6 | 0.16 | −2.5 | 0.0096 |
| P14780^{c} | Matrix metalloproteinase-9 (SEQ ID NO: 23) | −1.8 | 0.18 | −3.8 | 0.018 |
| Q9BXP8^{c} | Pappalysin-2 (SEQ ID NO: 38) | −1.0 | | 6 | 0.016 |
| P13796 | Plastin-2 (SEQ ID NO: 24) | −3.3 | <0.0001 | −3 | <0.0001 |
| P07737 | Profilin-1 (SEQ ID NO: 25) | −1.0 | 0.94 | −9.2 | <0.0001 |
| p11464^{b, c} | Pregnancy-specific beta-1-glycoprotein (SEQ ID | −1.2 | 0.02 | −2.1 | <0.0001 |
| | NO: 26) | | | | |
| P23470 | Receptor-type tyrosine-protein phosphatase gamma | −5.1 | 0.03 | −5.5 | 0.025 |
| | (SEQ ID NO: 27) | | | | |
| P20742 | Pregnancy zone protein (SEQ ID NO: 28) | −1.8 | <0.0001 | −1.8 | <0.0001 |
| P02753^{c} | Plasma retinol-binding protein (SEQ ID NO: 29) | −1.0 | 0.72 | 1.8 | <0.0001 |
| Q9H299 | SH3 domain-binding glutamic acid-rich-like protein 3 | −1.5 | 0.32 | −10.7 | 0.0003 |
| | (SEQ ID NO: 30) | | | | |
| P37802 | Transgelin-2 (SEQ ID NO: 31) | −1.3 | 0.44 | −16.9 | <0.0001 |
| Q9Y490 | Talin-1 (SEQ ID NO: 32) | 1.1 | 0.57 | −35.3 | <0.0001 |
| P67936 | Tropomyosin alpha-4 chain (SEQ ID NO: 33) | 1.4 | 0.24 | −16 | <0.0001 |
| Q6EMK4 | Vasorin (SEQ ID NO: 34) | 2.6 | 0.092 | 2.8 | 0.038 |
| P35916^{c} | Vascular endothelial growth factor receptor 3 (SEQ | −8.4 | 0.0018 | −9 | 0.0013 |
| | ID NO: 35) | | | | |
| P18206 | Vinculin (SEQ ID NO: 36) | −1.9 | 0.14 | −3.4 | 0.017 |
| P04275 | von Willebrand factor (SEQ ID NO: 37) | 1.1 | 0.21 | 2 | <0.0001 |

^{a}The fold expression change of protein was quantitated using the formula described in ref. [ ]. Proteins with significant (p-value <= 0.05 as highlighted in bold font and a fold change of >=±1.5) differential expression in any pair-wise comparisons are listed in the above table with their Swiss-Prot accessions.
^{b}Proteins that shared significant sequence homology are collapsed and treated as a single entry.
^{c}Proteins that were ran in immunoassay

Fourteen of these potential biomarkers were selected for further validation by immunoassay, based on statistical significance and/or potential clinical relevance. Measured protein concentrations from 118 subjects were log-transformed and compared between control and PE using an ANOVA test. The mean concentration of each protein in both groups was transformed back to original units (ng/ml, harmonic mean) for presentation. As summarized in Table 3a below, there were statistically significant differences in concentrations of 6 out of the 14 candidate proteins among women with and without PE. Cystatin-C, Endoglin, Fibronectin, Apolipoprotein C-III, Choriogonadotropin subunit beta and Pappalysin-2 had significantly higher concentrations in maternal serum from women with PE. Matrix metalloproteinase-9 is significantly lower in PE.

TABLE 3a
Differences in 14 candidate protein biomarkers between serum samples obtained from
women with pre-eclampsia versus those without pre-eclampsia.

| | Single Protein Comparisons | | |
|---|---|---|---|
| | Harmonic Mean Value for Each Group | | |
| | PE | No PE | PE vs. No PE |
| Protein | n = 58 | n = 58 | p-value* |
| Apolipoprotein B-100 (SEQ ID NO: 3) | 18255920.57 | 17929645.97 | 0.9605 |
| Cystatin-C (SEQ ID NO: 11) | 2093.82 | 1394.37 | [symbol] |
| Endoglin (SEQ ID NO: 12) | 96.54 | 48.48 | [symbol] |
| Fibronectin (SEQ ID NO: 15) | 1024791.77 | 178377.27 | [symbol] |
| Plasma retinol-binding protein (SEQ ID NO: 29) | 20537.34 | 14262.86 | 0.0221 |
| Apolipoprotein C-III (SEQ ID NO: 5) | 137310.49 | 73136.88 | [symbol] |
| Chorionic somatomammotropin hormone | 40538.20 | 46674.43 | 0.6140 |
| (SEQ ID NO: 10) | | | |
| Choriogonadotropin subunit beta (SEQ ID | 1799.66 | 835.22 | [symbol] |
| NO: 8) | | | |
| Pappalysin-2 (SEQ ID NO: 38) | 736.84 | 72.00 | [symbol] |
| Vascular endothelial growth factor receptor 3 | 50.61 | 52.48 | 0.8081 |
| (SEQ ID NO: 35 | | | |
| Histidine-rich glycoprotein (SEQ ID NO: 19) | 222957.00 | 126733.43 | 0.0182 |
| Insulin-like growth factor-binding protein 2 | 109.96 | 80.48 | 0.0098 |
| (SEQ ID NO: 20) | | | |
| Matrix metalloproteinase-9 (SEQ ID NO: 23) | 226.59 | 386.23 | 0.0171 |
| Pregnancy-specific beta-1-glycoprotein 1 | 35913.88 | 26118.50 | 0.2546 |
| (SEQ ID NO: 26) | | | |

p-value from one-way analysis of variance are on log-transformed data. Bold italics indicate statistically
significant differences between groups after Bonferroni adjustment for multiple comparisons applied

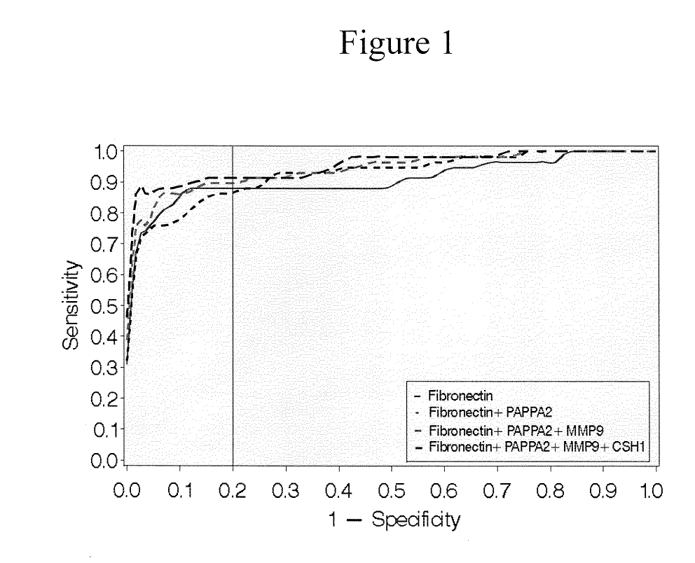

Simple logistic regression models (Hosmer and Lemeshow 2000) with a binary dependent variable designating PE status (1=PE n=60, 0=Control n=58) were fit for each biomarker individually. The predicted values from these models were used to create Receiver Operating Characteristic (ROC) curves (Pepe 2003). ROC curves are plots of the true positive fraction of a test (sensitivity) versus the false positive fraction (1-specificity) across the entire continuum of observed values. The area under the curve should be between 0.5 (poor discriminant) to 1.0 (perfect discriminant), and can be expressed probabilistically as the probability that a randomly selected pair of PE and control subjects is correctly classified. Standard errors for the AUROC were conducted based on percentiles of bootstrapped distributions (Pepe 2003). Table 3b summarizes the area under the entire receiver operating characteristic curve (AUROC) and 95% confidence intervals (CI) for the 14 potential biomarkers for PE. Fibronectin, Pappalysin-2, Endoglin, Cystatin-C and Apolipoprotein C-III had the best classification performance with AUROCs of 0.91, 0.89, 0.86, 0.77 and 0.76, respectively. As illustrated in FIG. 1, a three-analyte model including Fibronectin, Pappalysin-2 and Matrix metalloproteinase-9 had an improved AUROC of 0.944 (95% CI 0.90-0.98).

TABLE 3b
Performance of 14 candidate protein biomarkers, individually and
in combination, for classifying samples with or without pre-eclampsia.

| | | |
|---|---|---|
| | Area Under | |
| | ROC Curve | 95% CI for |
| Marker | (AUROC) | AUROC |

Individual Proteins

| | | |
|---|---|---|
| Apolipoprotein B-100 (SEQ ID NO: 3) | 0.356 | (0.23-0.44) |
| Cystatin-C (SEQ ID NO: 11) | 0.768 | (0.68-0.86) |
| Endoglin (SEQ ID NO: 12) | 0.857 | (0.79-0.92) |
| Fibronectin (SEQ ID NO: 15) | 0.909 | (0.85-0.97) |
| Plasma retinal-binding protein | 0.683 | (0.58-0.78) |
| (SEQ ID NO: 29) | | |
| Apolipoprotein C-III (SEQ ID NO: 5) | 0.762 | (0.67-0.81) |
| Chorionic somatomammotropin hormone | 0.636 | (0.53-0.74) |
| (SEQ ID NO: 10) | | |
| Choriogonadotropin subunit beta | 0.687 | (0.60-0.79) |
| (SEQ ID NO: 8) | | |
| Pappalysin-2 (SEQ ID NO: 38) | 0.889 | (0.83-0.95) |
| Vascular endothelial growth factor receptor 3 | 0.550 | (0.45-0.66) |
| (SEQ ID NO: 35 | | |
| Histidine-rich glycoprotein (SEQ ID NO: 19) | 0.649 | (0.55-0.75) |
| Insulin-like growth factor-binding protein 2 | 0.670 | (0.57-0.77) |
| (SEQ ID NO: 20) | | |
| Matrix metalloproteinase-9 (SEQ ID NO: 23) | 0.749 | (0.66-0.84) |
| Pregnancy-specific beta-1-glycoprotein 1 | 0.602 | (0.50-0.71) |
| (SEQ ID NO: 26) | | |

Protein Combinations

| | | |
|---|---|---|
| Fibro + PAPPA2 | 0.927 | (0.88-0.97) |
| Fibro + PAPPA2 + MMP9 | 0.944 | (0.90-0.98) |
| Fibro + PAPPA2 + MMP9 + CSH1 | 0.956 | (0.92-0.99) |

### Example 2

### Identification of Maternal Serum Biomarkers of Pre-Eclampsia During Early Gestation

Experimental Methods

Sample Collection and Processing: A total of 169 human subjects (control n=96, mild PE n=33 and severe PE n=40) were identified prospectively and given informed consent to participate in the study. The mean gestational age of the women at the collection was 10.1±1.3 weeks. All the samples were allowed to clot for 30 min., spun down at 5000 g, supernatant was collected and stored in −80° C. until further processing. Pre-eclampsia was defined as (ACOG criteria) systolic blood pressure of >140 mmHg or diastolic blood pressure >90 mmHg on at least two occasions, 4 hours to 1 week apart and protenuria (>300 mg in a 24 hour urine collection or 2+ on dip stick measurement). Severe pre-eclampsia is defined as systolic blood pressure of >160 mmHg, diastolic blood pressure>110 mmHg and/or proteinuria (>300 mg or 3+ on dip stick measurement).

MudPIT analysis, Enzyme-Linked Immunosorbent Assay (ELISA), statistical analysis of MudPIT data, and statistical analysis of ELISA data were performed as in Example 1.

Results

A total of 457 unique serum proteins were identified in this study. 45 differentially abundant maternal serum proteins in pre-eclampsia are summarized in Table 4 below. Independent pair-wise differences in abundance between samples were performed for each protein from women with and without PE. Proteins with a relative expression change of >1.5 fold and passes the chi-square test with a p-value<0.05 in any of the comparisons are considered as potentially differentially expressed between the samples. These included pregnancy proteins such as Choriogonadotropin subunit beta and Pappalysin-2, and extracellular matrix signaling factors such as Fibronectin and Matrix metalloproteinase-9.

TABLE 4a
Potential biomarkers for the detection of preeclampsia at gestational age of 9-11 weeks

| | | Fold Change^{a} | | 2 × 2 Chi-square p-value | |
|---|---|---|---|---|---|
| Swiss-Prot | | Mild PE vs. | Severe PE | Mild PE vs. | Severe PE |
| Accession | Protein name | Control | vs. Control | Control | vs. Control |
| P08697^{c} | Alpha-2-antiplasmin (SEQ ID NO: 39) | 1.8 | 2.4 | 0.0005 | <0.0001 |
| P60709 | Actin (SEQ ID NO: 40) | 1.7 | 1.6 | 0.0037 | 0.017 |
| P43652 | Afamin (SEQ ID NO: 41) | 1.5 | 1.7 | <0.0001 | <0.0001 |
| P01008 | Antithrombin-III (SEQ ID NO: 42) | 2.3 | 2.1 | <0.0001 | <0.0001 |
| P02652^{c} | Apolipoprotein A-II (SEQ ID NO: 43) | 5.7 | 5 | <0.0001 | <0.0001 |
| Q9NTQ4 | Attractin (SEQ ID NO: 44) | 2.5 | 2.4 | <0.0001 | <0.0001 |
| P61769^{c} | Beta-2-microglobulin (SEQ ID NO: 45) | 3.5 | 4.3 | 0.04 | 0.0083 |
| Q15582 | Transforming growth factor-beta-induced protein ig-h3 (SEQ | 1.2 | 1.7 | 0.43 | 0.034 |
| | ID NO: 46) | | | | |
| P04003 | C4b-binding protein alpha chain (SEQ ID NO: 47) | −1.6 | −1.6 | <0.0001 | <0.0001 |
| P07339^{c} | Cathepsin D (SEQ ID NO: 7) | 6.8 | 4.4 | 0.003 | 0.029 |
| Q96IY4 | Carboxypeptidase B2 (SEQ ID NO: 48) | 1.7 | 2.2 | 0.015 | 0.0004 |
| P00746^{c} | Complement factor D (SEQ ID NO: 49) | 5.6 | 4.6 | <0.0001 | 0.0003 |
| Q9NQ79 | Cartilage acidic protein 1 (SEQ ID NO: 50) | 3.1 | 2.7 | .0071 | 0.024 |
| P09172 | Dopamine beta-hydroxylase (SEQ ID NO: 51) | 1 | 1.8 | 0.84 | 0.03 |
| P05160 | Coagulation factor XIII B chain (SEQ ID NO: 52) | 2.6 | 2.4 | 0.0009 | 0.0035 |
| P02671 | Fibrinogen alpha chain (SEQ ID NO: 53) | 1.5 | 2.6 | 0.15 | 0.0001 |
| Q8IVI8^{c} | Fibronectin (SEQ ID NO: 15) | 1.6 | 2 | <0.0001 | <0.0001 |
| P21333 | Filamin-A (SEQ ID NO: 16) | 3.3 | 3.6 | 0.0004 | 0.0001 |
| P52566 | Rho GDP-dissociation inhibitor 2 (SEQ ID NO: 54) | 3 | 4.9 | 0.063 | 0.0031 |
| P07359 | Platelet glycoprotein lb alpha chain (SEQ ID NO: 55) | 1.8 | 2.3 | 0.12 | 0.031 |
| P00739 | Haptoglobin-related protein (SEQ ID NO: 56) | 1.7 | 1.7 | <0.0001 | <0.0001 |
| P18428^{c} | Lipopolysaccharide-binding protein (SEQ ID NO: 22) | 13.3 | 8.1 | <0.0001 | <0.0001 |
| P02753^{c} | Plasma retinol-binding protein (SEQ ID NO: 29) | 1.6 | 1.5 | <0.0001 | 0.0001 |
| P02775 | Platelet basic protein (SEQ ID NO: 57) | 1.5 | 1.6 | 0.0045 | 0.0002 |
| P37802 | Transgelin-2 (SEQ ID NO: 31) | 2.2 | 2.3 | 0.044 | 0.031 |
| Q9H4B7 | Tubulin beta-1 chain (SEQ ID NO: 58) | 4 | 3.8 | 0.014 | 0.024 |
| Q9Y490 | Talin-1 (SEQ ID NO: 32) | 2.7 | 2.9 | 0.0014 | 0.0007 |
| P62328 | Thymosin beta-4 (SEQ ID NO: 59) | 1.5 | 4.3 | 0.5 | 0.0024 |
| Q6EMK4^{c} | Vasorin (SEQ ID NO: 34) | 4.6 | 3.9 | 0.0009 | 0.0066 |
| P19320^{c} | Vascular cell adhesion protein 1 (SEQ ID NO: 60) | 5.1 | 3.3 | 0.0031 | 0.038 |
| P04275 | von Willebrand factor (SEQ ID NO: 37) | 2.6 | 2.2 | <0.0001 | <0.0001 |
| P25311 | Zinc-alpha-2-glycoprotein (SEQ ID NO: 61) | −5.6 | −5.1 | <0.0001 | <0.0001 |
| P01023^{c} | Alpha-2-macroglobulin (SEQ ID NO: 62) | −1.78 | −2.12 | <0.0001 | <0.0001 |
| Q13787^{c} | Apolipoprotein B-100 (SEQ ID NO: 3) | −1.48 | −1.49 | <0.0001 | <0.0001 |
| P02656^{c} | Apolipoprotein C-III (SEQ ID NO: 5) | 1.93 | 1.89 | 0.009 | 0.012 |
| P01233^{c} | Choriogonadotropin subunit beta (SEQ ID NO: 8) | 1.46 | 1.4 | 0.0065 | 0.0127 |
| P01243^{b, c} | Chorionic somatomammotropin hormone (SEQ ID NO: 10) | 2.02 | −1.66 | 0.299 | N/A |
| P01034^{c} | Cystatin-C (SEQ ID NO: 11) | −1.69 | −1.43 | 0.515 | 0.757 |
| P17813^{c} | Endoglin (SEQ ID NO: 12) | −1.69 | 1.14 | 1 | 1 |
| P14780^{c} | Matrix metalloproteinase-9 (SEQ ID NO: 23) | 1.38 | 1.52 | 0.594 | 0.44 |
| Q13219^{c} | Pappalysin-1 (SEQ ID NO: 63) | −1.82 | −1.26 | 0.628 | 0.531 |
| Q9P1W5^{c} | Pregnancy-specific beta-1-glycoprotein 1 (SEQ ID NO: 26) | 1.01 | 1.06 | 0.922 | 0.708 |
| P35916^{c} | Vascular endothelial growth factor receptor 3 | −1.69 | −1.15 | 1 | 1 |
| | (SEQ ID NO: 35) | | | | |
| P02741^{c} | C-reactive protein (SEQ ID NO: 64) | −1 | −1.05 | 0.8994 | 0.9966 |
| P02743^{c} | Serum amyloid P-component (SEQ ID NO: 65) | 1.17 | 2.57 | 0.6 | 0.0068 |

^{a}The fold expression change of protein was quantitated using the formula described in ref. [ ]
^{b}Proteins that showed significant fold change but did not reach statistical significance due to small number of spectral counts
^{c}Proteins that were run in immunoassay
Bold values in p-value columns are <0.05

TABLE 4b
Potential biomarkers for the detection of preeclampsia at gestational age of 10-14
weeks

| | | Fold Change | | | P-value | | |
|---|---|---|---|---|---|---|---|
| Swiss | | Mild PE | Severe | Severe | Mild PE | Severe | Severe |
| Prot | | vs. | PE vs. | PE vs. | vs. | PE vs. | PE vs. |
| Accession | Description | Control | Control | Mild PE | Control | Control | Mild PE |
| Q16853 | Membrane copper amine | 2.6 | 5.8 | 2.2 | | 1.1E−02 | 1.4E−01 |
| | oxidase (SEQ ID NO: 103) | | | | | | |
| P02741 | C-reactive protein (SEQ ID | 5.9 | 5.7 | −1.0 | 1.0E−03 | 1.7E−03 | 8.9E−01 |
| | NO: 64) | | | | | | |
| P02743 | Serum amyloid P-component | 2.4 | 5.3 | 2.2 | 6.2E−02 | 1.1E−05 | 5.7E−03 |
| | (SEQ ID NO: 65) | | | | | | |
| Q9BXR6 | Complement factor H-related | 5.1 | 5.2 | 1.0 | 2.1E−02 | 2.0E−02 | 9.8E−01 |
| | protein 5 (SEQ ID NO: 67) | | | | | | |
| Q9Y6V0 | Protein piccolo (SEQ ID NO: 68) | −1.3 | 4.6 | 5.9 | | 3.8E−02 | 1.5E−02 |
| P12955 | Xaa-Pro dipeptidase (SEQ ID | −1.3 | 4.6 | 5.9 | | 3.8E−02 | 1.5E−02 |
| | NO: 69) | | | | | | |
| Q9UPA5 | Protein bassoon (SEQ ID | −1.3 | 3.9 | 5.1 | | 7.2E−02 | 3.0E−02 |
| | NO: 70) | | | | | | |
| Q9H4B7 | Tubulin beta-1 chain (SEQ ID | 3.6 | 3.4 | −1.1 | 1.1E−02 | 1.8E−02 | 8.8E−01 |
| | NO: 58) | | | | | | |
| Q14118 | Dystroglycan (SEQ ID NO: 71) | 4.5 | 2.7 | −1.7 | 3.9E−02 | | 5.1E−01 |
| P04040 | Catalase (SEQ ID NO: 72) | −1.8 | 2.4 | 4.3 | 3.5E−01 | 2.3E−02 | 4.9E−04 |
| P00738 | | | | | | | |
| P00739 | Haptoglobin, Haptoglobin | 2.2 | 2.1 | −1.0 | 2.9E−34 | 2.4E−32 | 6.9E−01 |
| | related protein (SEQ ID NO: 56) | | | | | | |
| P00915 | Carbonic anhydrase 1 (SEQ ID | 1.1 | 2.0 | 1.7 | 6.0E−01 | 2.5E−03 | 5.8E−03 |
| | NO: 73) | | | | | | |
| P05362 | Intercellular adhesion | 2.6 | 1.9 | −1.4 | 4.7E−02 | 2.5E−01 | 4.1E−01 |
| | molecule 1 (SEQ ID NO: 74) | | | | | | |
| P02787 | Serotransferrin (SEQ ID NO: 75) | 1.6 | 1.9 | 1.2 | 1.5E−10 | 1.1E−18 | 5.9E−03 |
| Q08380 | Galectin-3-binding protein | 1.9 | 1.9 | −1.0 | 1.7E−02 | 1.9E−02 | 9.6E−01 |
| | (SEQ ID NO: 76) | | | | | | |
| P62328 | Thymosin beta-4 (SEQ ID | −1.6 | 1.8 | 2.9 | 3.1E−01 | 9.3E−02 | 4.8E−03 |
| | NO: 59) | | | | | | |
| P02753 | Plasma retinol-binding protein | 1.9 | 1.7 | −1.1 | 7.5E−11 | 1.4E−08 | 3.5E−01 |
| | (SEQ ID NO: 29) | | | | | | |
| P01233 | Choriogonadotropin subunit | , | 1.7 | −1.0 | 6.5E−03 | 1.3E−02 | 8.0E−01 |
| | beta (SEQ ID NO: 8) | | | | | | |
| P32119 | Peroxiredoxin-2 (SEQ ID | −1.1 | 1.6 | 1.7 | 7.8E−01 | 5.8E−02 | 1.9E−02 |
| | NO: 77) | | | | | | |
| P09172 | Dopamine beta-hydroxylase | −1.3 | 1.3 | 1.7 | 2.8E−01 | 2.5E−01 | 1.7E−02 |
| | (SEQ ID NO: 51) | | | | | | |
| P20742 | Pregnancy zone protein (SEQ | −1.3 | 1.3 | 1.6 | 5.8E−01 | 2.5E−03 | 7.5E−10 |
| | ID NO: 28) | | | | | | |
| P07738 | Bisphosphoglycerate mutase | −3.6 | 1.2 | 4.2 | 9.2E−02 | 6.9E−01 | 2.1E−02 |
| | (SEQ ID NO: 78 | | | | | | |
| P08185 | Corticosteroid-binding | 1.6 | 1.1 | −1.4 | 3.3E−02 | 6.9E−01 | 5.9E−02 |
| | globulin (SEQ ID NO: 79 | | | | | | |
| P00918 | Carbonic anhydrase 2 (SEQ ID | −1.8 | 1.0 | 1.8 | 4.0E−02 | 9.4E−01 | 2.6E−02 |
| | NO: 80 | | | | | | |
| P18428 | Lipopolysaccharide-binding | 1.3 | −1.2 | −1.6 | 2.0E−01 | 4.3E−01 | 2.8E−02 |
| | protein (SEQ ID NO: 22) | | | | | | |
| P25054 | Adenomatous polyposis coli | 5.7 | −1.3 | −7.3 | 1.1E−02 | | 7.8E−03 |
| | protein (SEQ ID NO: 81) | | | | | | |
| P22064 | Latent-transforming growth | 4.6 | −1.3 | −5.8 | 9.5E−03 | | 3.4E−03 |
| | factor beta-binding (SEQ ID | | | | | | |
| | NO: 82) | | | | | | |
| P02671 | Fibrinogen alpha chain (SEQ | −2.3 | −1.3 | 1.8 | 5.0E−06 | 1.2E−01 | 1.6E−03 |
| | ID NO: 53) | | | | | | |
| P00740 | Coagulation factor IX (SEQ ID | −1.7 | −1.4 | 1.2 | 2.3E−03 | 2.7E−02 | 3.8E−01 |
| | NO: 83) | | | | | | |
| Q04756 | Hepatocyte growth factor | −1.6 | −1.5 | 1.1 | 3.1E−02 | 7.7E−02 | 6.8E−01 |
| | activator (SEQ ID NO: 84) | | | | | | |
| P02747 | Complement C1q | −1.8 | −1.5 | 1.2 | 6.3E−04 | 1.8E−02 | 2.7E−01 |
| | subcomponent subunit C (SEQ | | | | | | |
| | ID NO: 85) | | | | | | |
| P02746 | Complement C1q | −1.7 | −1.5 | 1.1 | 4.0E−03 | 1.9E−02 | 5.9E−01 |
| | subcomponent subunit B (SEQ | | | | | | |
| | ID NO: 86) | | | | | | |
| P49747 | Cartilage oligomeric matrix | −1.4 | −1.5 | −1.1 | 7.9E−02 | 2.6E−02 | 6.1E−01 |
| | protein (SEQ ID NO: 87) | | | | | | |
| P13796 | Plastin-2 (SEQ ID NO: 24) | −1.7 | −1.5 | 1.1 | 5.2E−03 | 2.6E−02 | 5.6E−01 |
| P04275 | von Willebrand factor (SEQ ID | −1.3 | −1.5 | −1.2 | 6.4E−03 | 5.0E−05 | 1.6E−01 |
| | NO: 37) | | | | | | |
| P60709, | Actin, cytoplasmic 1, Actin, | −1.3 | −1.6 | −1.2 | 4.2E−02 | 1.3E−03 | 2.1E−01 |
| P63267 | gamma-enteric smooth | | | | | | |
| | muscle (SEQ ID NO: 40), (SEQ | | | | | | |
| | ID NO: 88) | | | | | | |
| P10721 | Mast/stem cell growth factor | −3.1 | −1.6 | 2.0 | 3.9E−02 | 3.2E−01 | 2.2E−01 |
| | receptor (SEQ ID NO: 89) | | | | | | |
| P40197 | Platelet glycoprotein V (SEQ | −1.8 | −1.6 | 1.1 | 4.0E−02 | 8.7E−02 | 7.2E−01 |
| | ID NO: 90) | | | | | | |
| Q8WZ75 | Roundabout homolog 4 (SEQ | −7.5 | −1.8 | 4.3 | 6.9E−03 | 3.5E−01 | |
| | ID NO: 91) | | | | | | |
| Q16610 | Extracellular matrix protein 1 | −1.1 | −1.8 | −1.6 | 5.3E−01 | 4.5E−03 | 2.0E−02 |
| | (SEQ ID NO: 92) | | | | | | |
| P02745 | Complement C1q | −2.2 | −1.8 | 1.2 | 4.0E−02 | 1.1E−01 | 6.3E−01 |
| | subcomponent subunit A (SEQ | | | | | | |
| | ID NO: 93) | | | | | | |
| P55058 | Phospholipid transfer protein | −3.7 | −1.9 | 1.9 | 2.6E−02 | 2.0E−01 | 2.9E−01 |
| | (SEQ ID NO: 94) | | | | | | |
| Q76LX8 | ADAMTS-13 (SEQ ID NO: 95) | −1.5 | −1.9 | −1.3 | 2.0E−01 | 3.8E−02 | 4.1E−01 |
| P05155 | Plasma protease C1 inhibitor | −2.5 | −1.9 | 1.3 | 3.0E−12 | 9.1E−08 | 8.3E−02 |
| | (SEQ ID NO: 96) | | | | | | |
| Q13790 | Apolipoprotein F (SEQ ID | −1.6 | −2.2 | −1.4 | 2.1E−01 | 4.0E−02 | 3.9E−01 |
| | NO: 97) | | | | | | |
| Q99784 | Noelin (SEQ ID NO: 98) | −2.7 | −2.4 | 1.2 | 1.7E−02 | 3.6E−02 | 7.6E−01 |
| O75015 | Low affinity immunoglobulin | −1.4 | −3.1 | −2.1 | 4.4E−01 | 4.0E−02 | 2.3E−01 |
| | gamma Fc region receptor | | | | | | |
| | (SEQ ID NO: 99) | | | | | | |
| Q16208 | CD44 antigen (SEQ ID NO: 100) | −1.2 | −3.4 | −2.8 | 7.1E−01 | 2.3E−02 | 5.7E−02 |
| P22897 | Macrophage mannose | −2.2 | −4.1 | −1.9 | 1.9E−01 | 4.9E−02 | |
| | receptor 1 (SEQ ID NO: 101) | | | | | | |
| P01243 | Chorionic | −1.9 | −6.4 | −3.4 | 3.1E−01 | 2.3E−02 | |
| | somatomammotropin | | | | | | |
| | hormone (SEQ ID NO: 10) | | | | | | |
| P02675 | Fibrinogen beta chain (SEQ ID | −10.6 | −10.4 | 1.0 | 5.7E−04 | 6.2E−04 | |
| | NO: 102) | | | | | | |

Twenty three of these potential biomarkers were selected for further validation by immunoassay, based on statistical significance and/or potential clinical relevance. Measured protein concentrations from 169 subjects were log-transformed and compared between control and PE using an ANOVA test. The mean concentration of each protein in both groups was transformed back to original units (ng/ml, harmonic mean) for presentation. As summarized in Table 5a below, there were statistically significant differences in concentrations of 2 out of the 23 candidate proteins among women with and without PE. Complement factor D and Vascular cell adhesion protein 1 had significantly higher concentrations in maternal serum from women with PE. As seen in Table 5b, Pappalysin-1 was relatively low among women with PE in the comparison of severe PE and control.

TABLE 5a
Differences in 23 candidate protein biomarkers in serum samples from women in first
trimester with pre-eclampsia and without pre-eclampsia

| | Single Protein Comparisons | | |
|---|---|---|---|
| | Harmonic Mean Value for Each Group | | |
| | | | PE vs. No |
| | PE | No PE | PE |
| Protein | n = 73 | n = 96 | p-value* |
| Apolipoprotein A-II (SEQ ID NO: 43) | 424119.61 | 436347.53 | 0.4836 |
| Beta-2-microglobulin (SEQ ID NO: 45) | 1203.01 | 1102.85 | 0.0158 |
| Complement factor D (SEQ ID NO: 49) | 2201.04 | 1914.27 | [symbol] |
| Vasorin (SEQ ID NO: 34) | 7758.79 | 7061.72 | 0.0207 |
| Alpha-2-antiplasmin (SEQ ID NO: 39) | 1020.85 | 830.61 | 0.3814 |
| Apolipoprotein C-III (SEQ ID NO: 5) | 52994.68 | 52686.57 | 0.9151 |
| Vascular cell adhesion protein 1 (SEQ ID | 12415.61 | 10793.85 | [symbol] |
| NO: 60) | | | |
| Alpha-2-macroglobulin (SEQ ID NO: 62) | 1733583.31 | 1864547.21 | 0.233 |
| Pappalysin-1 (SEQ ID NO: 63) | 400.55 | 1935.27 | 0.0091 |
| Apolipoprotein B-100 (SEQ ID NO: 3) | 20721328.71 | 20942859.27 | 0.8119 |
| Cystatin-C (SEQ ID NO: 11) | 1558.57 | 1450.36 | 0.0033 |
| Endoglin (SEQ ID NO: 12) | 33.94 | 35.64 | 0.124 |
| Fibronectin (SEQ ID NO: 15) | 703148.86 | 685657.84 | 0.8633 |
| Plasma retinol-binding protein (SEQ ID NO: 29) | 21801.27 | 21941.98 | 0.8071 |
| Chorionic somatomammotropin hormone | 1775.39 | 2455.75 | 0.3207 |
| (SEQ ID NO: 10) | | | |
| Choriogonadotropin subunit beta (SEQ ID | 6010.62 | 5333.17 | 0.2031 |
| NO: 8) | | | |
| Vascular endothelial growth factor receptor 3 | 17.86 | 18.59 | 0.5362 |
| (SEQ ID NO: 35) | | | |
| Lipopolysaccharide-binding protein (SEQ ID | 44265.63 | 40592.77 | 0.3203 |
| NO: 22) | | | |
| Pregnancy-specific beta-1-glycoprotein 1 | 5177.94 | 5595.67 | 0.6162 |
| (SEQ ID NO: 26) | | | |
| Matrix metalloproteinase-9 (SEQ ID NO: 23) | 501.45 | 544.94 | 0.3783 |
| Cathepsin D (SEQ ID NO: 7) | 3372.27 | 3019.25 | 0.4423 |
| C-reactive protein (SEQ ID NO: 64) | 1760.81 | 1483.24 | 0.2994 |
| Serum amyloid P-component (SEQ ID NO: 65) | 24406.15 | 23469.36 | 0.4374 |

p-value from one-way analysis of variance are on log-transformed data. Bold italics indicate statistically
significant differences between groups after Bonferroni adjustment for multiple comparisons applied

TABLE 5b
Differences in 23 candidate protein biomarkers in serum samples from women in first
trimester with severe pre-eclampsia compared to controls

| | Single Protein Comparisons | | |
|---|---|---|---|
| | Harmonic Mean Value for Each Group | | |
| | | | Severe PE |
| | Severe PE | Control | vs. Control |
| Protein | n = 40 | n = 96 | p-value* |
| Apolipoprotein A-II (SEQ ID NO: 43) | 414589.35 | 436347.53 | 0.3113 |
| Beta-2-microglobulin (SEQ ID NO: 45) | 1221.49 | 1102.85 | 0.0238 |
| Complement factor D (SEQ ID NO: 49) | 2125.16 | 1914.27 | 0.0209 |
| Vasorin (SEQ ID NO: 34) | 7356.94 | 7061.72 | 0.3956 |
| Alpha-2-antiplasmin (SEQ ID NO: 39) | 972.32 | 830.61 | 0.6003 |
| Apolipoprotein C-III (SEQ ID NO: 5) | 51238.62 | 52686.57 | 0.6669 |
| Vascular cell adhesion protein 1 (SEQ ID | 11978.60 | 10793.85 | 0.0355 |
| NO: 60) | | | |
| Alpha-2-macroglobulin (SEQ ID NO: 62) | 1804294.11 | 1864547.21 | 0.4176 |
| Pappalysin-1 (SEQ ID NO: 63) | 181.32 | 1935.27 | [symbol] |
| Apolipoprotein B-100 (SEQ ID NO: 3) | 21038246.48 | 20942859.27 | 0.9344 |
| Cystatin-C (SEQ ID NO: 11) | 1569.99 | 1450.36 | 0.0099 |
| Endoglin (SEQ ID NO: 12) | 33.72 | 35.64 | 0.1623 |
| Fibronectin (SEQ ID NO: 15) | 779801.36 | 685657.84 | 0.4855 |
| Plasma retinol-binding protein (SEQ ID NO: 29) | 21146.57 | 21941.98 | 0.2714 |
| Chorionic somatomammotropin hormone | 1387.86 | 2455.75 | 0.1938 |
| (SEQ ID NO: 10) | | | |
| Choriogonadotropin subunit beta (SEQ ID | 5490.13 | 5333.17 | 0.8047 |
| NO: 8) | | | |
| Vascular endothelial growth factor receptor 3 | 18.08 | 18.59 | 0.7254 |
| (SEQ ID NO: 35) | | | |
| Lipopolysaccharide-binding protein (SEQ ID | 41060.55 | 40592.77 | 0.9814 |
| NO: 22) | | | |
| Pregnancy-specific beta-1-glycoprotein 1 | 5047.31 | 5595.67 | 0.5611 |
| (SEQ ID NO: 26) | | | |
| Matrix metalloproteinase-9 (SEQ ID NO: 23) | 458.81 | 544.94 | 0.7635 |
| Cathepsin D (SEQ ID NO: 7) | 3261.59 | 3019.25 | 0.7213 |
| Serum amyloid P-component (SEQ ID NO: 65) | 24046.97 | 23469.36 | 0.5732 |
| C-reactive protein (SEQ ID NO: 64) | 1994.80 | 1483.24 | 0.1215 |

Simple logistic regression models (Hosmer and Lemeshow 2000) with a binary dependent variable designating PE status (1=PE n=60, 0=Control n=58) were fit for each biomarker individually. The predicted values from these models were used to create Receiver Operating Characteristic (ROC) curves (Pepe 2003). ROC curves are plots of the true positive fraction of a test (sensitivity) versus the false positive fraction (1-specificity) across the entire continuum of observed values. The area under the curve should be between 0.5 (poor discriminant) to 1.0 (perfect discriminant), and can be expressed probabilistically as the probability that a randomly selected pair of PE and control subjects is correctly classified. Standard errors for the AUROC were conducted based on percentiles of bootstrapped distributions (Pepe 2003).

Table 6a summarizes the area under the entire receiver operating characteristic curve (AUROC) and 95% confidence intervals (CI) for the 23 potential biomarkers for classifying samples with PE (n=73) or without PE (n=96). Complement factor D (AUROC 0.67, 95% CI 0.59-0.75) and Pappalysin-1 (AUROCs of 0.66, 0.65) showed good classification ability. A Six-analyte model including Vascular cell adhesion protein 1, Endoglin, Complement factor D, Pappalysin-1, Choriogonadotropin subunit beta and Plasma retinol-binding protein had an improved AUROC of 0.77 (95% CI 0.70-0.84).

TABLE 6a
Performance of 23 candidate protein biomarkers, individually and in combination, for
classifying samples with or without pre-eclampsia

| | | |
|---|---|---|
| | Area Under ROC | 95% CI for |
| Marker | Curve (AUROC) | AUROC |

Individual Proteins

| | | |
|---|---|---|
| Apolipoprotein A-II (SEQ ID NO: 43) | 0.535 | (0.45-0.62) |
| Beta-2-microglobulin (SEQ ID NO: 45) | 0.604 | (0.52-0.69) |
| Complement factor D (SEQ ID NO: 49) | 0.669 | (0.59-0.75) |
| Vasorin (SEQ ID NO: 34) | 0.599 | (0.51-0.69) |
| Alpha-2-antiplasmin (SEQ ID NO: 39) | 0.514 | (0.45-0.62) |
| Apolipoprotein C-III (SEQ ID NO: 5) | 0.500 | (0.41-0.59) |
| Vascular cell adhesion protein 1 (SEQ ID NO: 60) | 0.653 | (0.57-0.74) |
| Alpha-2-macroglobulin (SEQ ID NO: 62) | 0.544 | (0.45-0.63) |
| Pappalysin-1 (SEQ ID NO: 63) | 0.663 | (0.58-0.75) |
| Apolipoprotein B-100 (SEQ ID NO: 3) | 0.517 | (0.43-0.61) |
| Cystatin-C (SEQ ID NO: 11) | 0.620 | (0.53-0.71) |
| Endoglin (SEQ ID NO: 12) | 0.576 | (0.49-0.66) |
| Fibronectin (SEQ ID NO: 15) | 0.515 | (0.42-0.60) |
| Plasma retinol-binding protein (SEQ ID NO: 29) | 0.505 | (0.42-0.59) |
| Chorionic somatomammotropin hormone (SEQ ID | 0.652 | (0.57-0.73) |
| NO: 10) | | |
| Choriogonadotropin subunit beta (SEQ ID NO: 8) | 0.532 | (0.44-0.62) |
| Vascular endothelial growth factor receptor 3 | 0.529 | (0.44-0.62) |
| (SEQ ID NO: 35) | | |
| Lipopolysaccharide-binding protein (SEQ ID | 0.541 | (0.45-0.63) |
| NO: 22) | | |
| Pregnancy-specific beta-1-glycoprotein 1 (SEQ ID | 0.621 | (0.54-0.71) |
| NO: 26) | | |
| Matrix metalloproteinase-9 (SEQ ID NO: 23) | 0.552 | (0.46-0.64) |
| Cathepsin D (SEQ ID NO: 7) | 0.544 | (0.45-0.63) |
| C-reactive protein (SEQ ID NO: 64) | 0.553 | (0.46-0.64) |
| Serum amyloid P-component (SEQ ID NO: 65) | 0.527 | (0.44-0.62) |

Protein Combinations

| | | |
|---|---|---|
| VCAM1 + Endoglin | 0.704 | (0.62-0.78) |
| VCAM1 + Endoglin + CFAD | 0.731 | (0.65-0.81) |
| VCAM1 + Endoglin + HCG + PAPPA1 | 0.749 | (0.67-0.82) |
| VCAM1 + Endoglin + HCG + PAPPA1 + CFAD | 0.756 | (0.68-0.83) |
| VCAM1 + Endoglin + HCG + PAPPA1 + CFAD + RBP | 0.771 | (0.70-0.84) |

Table 6b summarizes AUROC and 95% confidence intervals (CI) for 23 potential biomarkers for classifying samples with severe PE (n=40) versus control (n=96). Pappalysin-1 had the best classification performance (AUROC 0.68, 95% CI 0.58-0.79). A Five-analyte model including Pappalysin-1, C-reactive protein, Plasma retinol-binding protein, Beta-2-microglobulin and Vascular cell adhesion protein 1 had an improved AUROC of 0.75 (95% CI 0.68-0.83).

TABLE 6b
Performance of 23 candidate protein biomarkers,
individually and in combination, for classifying samples
with severe pre-eclampsia versus control

| | | |
|---|---|---|
| | Area Under | |
| | ROC Curve | 95% CI for |
| Marker | (AUROC) | AUROC |

Individual Proteins

| | | |
|---|---|---|
| Apolipoprotein A-II (SEQ ID NO: 43) | 0.556 | (0.45-0.67) |
| Beta-2-microglobulin (SEQ ID NO: 45) | 0.614 | (0.52-0.72) |
| Complement factor D (SEQ ID NO: 49) | 0.620 | (0.52-0.72) |
| Vasorin (SEQ ID NO: 34) | 0.536 | (0.43-0.64) |
| Alpha-2-antiplasmin (SEQ ID NO: 39) | 0.507 | (0.40-0.62) |
| Apolipoprotein C-III (SEQ ID NO: 5) | 0.526 | (0.43-0.64) |
| Vascular cell adhesion protein 1 (SEQ ID | 0.613 | (0.52-0.72) |
| NO: 60) | | |
| Alpha-2-macroglobulin (SEQ ID NO: 62) | 0.524 | (0.43-0.64) |
| Pappalysin-1 (SEQ ID NO: 63) | 0.684 | (0.58-0.79) |
| Apolipoprotein B-100 (SEQ ID NO: 3) | 0.511 | (0.42-0.61) |
| Cystatin-C (SEQ ID NO: 11) | 0.615 | (0.52-0.72) |
| Endoglin (SEQ ID NO: 12) | 0.582 | (0.49-0.69) |
| Fibronectin (SEQ ID NO: 15) | 0.566 | (0.47-0.78) |
| Plasma retinol-binding protein | 0.552 | (0.45-0.56) |
| (SEQ ID NO: 29) | | |
| Chorionic somatomammotropin hormone | 0.671 | (0.57-0.78) |
| (SEQ ID NO: 10) | | |
| Choriogonadotropin subunit beta | 0.490 | (0.40-0.61) |
| (SEQ ID NO: 8) | | |
| Vascular endothelial growth factor receptor 3 | 0.512 | (0.42-0.61) |
| (SEQ ID NO: 35) | | |
| Lipopolysaccharide-binding protein | 0.470 | (0.46-0.67) |
| (SEQ ID NO: 22) | | |
| Pregnancy-specific beta-1-glycoprotein 1 | 0.644 | (0.54-0.75) |
| (SEQ ID NO: 26) | | |
| Matrix metalloproteinase-9 | 0.541 | (0.44-0.64) |
| (SEQ ID NO: 23) | | |
| Cathepsin D (SEQ ID NO: 7) | 0.525 | (0.43-0.64) |
| Serum amyloid P-component | 0.522 | (0.42-0.63) |
| (SEQ ID NO: 65) | | |
| C-reactive protein (SEQ ID NO: 64) | 0.594 | (0.49-0.69) |

Protein Combinations

| | | |
|---|---|---|
| PAPPA1 + CRP | 0.712 | (0.62-0.81) |
| PAPPA + CRP + RBP | 0.726 | (0.63-0.82) |
| PAPPA + CRP + RBP + B2MG | 0.732 | (0.64-0.82) |

2. Discussion

We have utilized two comprehensive proteomic techniques to characterize maternal serum proteins among a cohort of women with and without PE.

Throughout the foregoing description the invention has been discussed with reference to certain embodiments, but it is not so limited. Indeed, various modifications of the invention in addition to those shown and described herein will become apparent to those skilled in the art from the foregoing description and fall within the scope of the appended claims.

All references cited throughout the description, and the references cited therein, are hereby expressly incorporated by reference in their entirety.

### Example 3

### Maternal Serum Biomarkers of Gestational Hypertension Distinct from Pre-Eclampsia

Study Design: To characterize maternal serum proteome profile in gestational hypertension (GH), or pregnancy induced hypertension (PIH), a total of 130 women from a prospective observational cohort were included in this study. Maternal serum samples were collected between 21 and 37 gestational weeks. GH and preeclampsia were classified by Working Group criteria (Am J Obstet Gynecol 2000; 183). Maternal serum proteome analysis was performed using multidimensional liquid chromatography tandem mass spectrometry (2D LC-MS/MS) and label-free quantification (spectral counting). Pair-wise comparison was performed using ×2 goodness-of-fit tests and adjusted for multiple comparisons via the false-discovery rate (FDR) method. Immunoassays were used for accurate quantification and evaluated using the Receiver Operating Characteristic (ROC) curves and logistic regression analysis.

Results: 14 women developed GH at a mean of 32 weeks gestation, 29 developed mild PE (mean 35 weeks), 29 developed severe PE (mean 31 weeks), and 58 remained normotensive and delivered at term. 2D-LC-MS-MS analysis of maternal sera identified 480 unique proteins for label-free quantification. Cluster analysis showed a unique cluster of proteins differentially expressed in PIH distinct from mild and severe PE. Label-free quantification identified 36 differentially expressed (p<0.05) proteins between patients with GH compared to PE. These included cytoskelatal proteins (talin, filamin A, tropomyosin alpha, actin aortic smooth muscle); placental proteins (PAPPA-2, HCG); and matrix proteins. Analysis of 17 potential biomarkers with specific immunoassays showed good discriminating capability between GH and PE (AUROC's 0.73 to 0.82). Multi-analyte analysis showed further increased the discriminant ability (AUROC>0.88).

TABLE 7
Serum proteins sharing significant changes between Pair-wise comparisons of
gestational hypertension, control, mild PE and severe PE samples

| | | Fold Change | | | Significance | | |
|---|---|---|---|---|---|---|---|
| | | GH | GH vs. | GH vs. | GH vs. | GH vs. | GH vs. |
| Swiss-Prot | | vs. | Mild PE | Severe | Control | Mild PE | Severe |
| Accession | Description | Control | levis | PE | P-val | P-val | PE P-val |
| P01034 | Cystatin-C (SEQ ID NO: 11) | 11.07 | 1.71 | 1.06 | 0.000 | 0.199 | 0.893 |
| P02763 | Alpha-1-acid glycoprotein 1 (SEQ ID NO: 104) | 5.22 | 5.33 | 1.36 | 0.028 | 0.027 | 0.742 |
| P61769 | Beta-2-microglobulin [Contains: Beta-2- | 5.22 | −1.09 | −1.15 | 0.028 | 0.844 | 0.729 |
| | microglobulin varian pl 5.3] (SEQ ID NO: 45) | | | | | | |
| P07339 | Cathepsin D (SEQ ID NO: 7) | 5.22 | 1.07 | −1.29 | 0.028 | 0.920 | 0.553 |
| P07942 | Laminin subunit beta-1 (SEQ ID NO: 21) | 3.55 | 1.39 | −2.73 | | | 0.041 |
| P17813 | Endoglin (SEQ ID NO: 12) | 2.71 | 2.77 | −3.84 | | | 0.009 |
| Q8IV18 | Fibronectin (SEQ ID NO: 15) | 2.09 | 1.01 | −5.22 | 0.000 | 0.894 | 0.000 |
| Q6EMK4 | Vasorin (SEQ ID NO: 34) | 2.03 | −1.28 | −1.40 | 0.217 | 0.550 | 0.391 |
| P01243 | Chorionic somatomammotropin hormone (SEQ | −2.00 | 1.44 | 1.09 | 0.009 | 0.283 | 0.808 |
| | ID NO: 10) | | | | | | |
| Q9H299 | SH3 domain binding glutamic acid-rich-like | −2.24 | −1.48 | 4.80 | 0.120 | 0.551 | |
| | protein 3 (SEQ ID NO: 30) | | | | | | |
| P33151 | Cadherin-5 (SEQ ID NO: 105) | −2.42 | −1.83 | −1.21 | 0.078 | 0.270 | 0.759 |
| P21333 | Filamin-A (SEQ ID NO: 16) | −2.48 | −2.57 | 12.12 | 0.003 | 0.002 | 0.000 |
| P07737 | Profilin-1 (SEQ ID NO: 25) | −2.55 | −2.50 | 3.61 | 0.006 | 0.007 | 0.030 |
| P07359 | Platelet glycoprotein lb alpha chain (SEQ ID | −2.76 | −1.82 | −3.55 | 0.058 | 0.346 | 0.009 |
| | NO: 55) | | | | | | |
| P02743 | Serum amyloid P-component (SEQ ID NO: 65) | −2.80 | 5.33 | −3.11 | 0.014 | 0.027 | 0.005 |
| P04075 | Fructose-bisphosphate aldolase A (SEQ ID | −2.95 | −1.39 | 1.68 | 0.009 | 0.472 | 0.486 |
| | NO: 106) | | | | | | |
| P37802 | Transgelin-2 (SEQ ID NO: 31) | −2.95 | −2.29 | 5.72 | 0.009 | 0.054 | 0.022 |
| P18206 | Vinculin (SEQ ID NO: 36) | −2.99 | −1.60 | 1.14 | 0.036 | 0.510 | |
| Q9NQ79 | Cartilage acidic protein 1 (SEQ ID NO: 50) | −3.21 | −2.49 | −1.29 | 0.022 | 0.094 | 0.731 |
| Q86SQ4 | Probable G-protein couple receptor 126 (SEQ | −3.26 | 1.07 | −4.37 | | | 0.065 |
| | ID NO: 107) | | | | | | |
| P13796 | Plastin-2 (SEQ ID NO: 24) | −3.31 | −1.00 | −1.12 | 0.000 | 0.977 | 0.715 |
| P67936 | Tropomyosin alpha-4 chain (SEQ ID NO: 33) | −3.33 | −4.70 | 4.80 | 0.007 | 0.000 | |
| Q6P3U9 | 14-3-3 protein zeta/delta (SEQ ID NO: 108) | −3.61 | −2.38 | 2.97 | 0.023 | 0.182 | |
| P23528 | Cofilin-1 (SEQ ID NO: 109) | −4.79 | −5.45 | 1.14 | 0.063 | 0.032 | |
| P04406 | Glyceraldehyde-3-phosphate dehydrogenase | −4.79 | 1.07 | 1.14 | 0.063 | | |
| | (SEQ ID NO: 110) | | | | | | |
| Q9UJJ9 | N-acetylglucosamine-1-phosphotransferase | −4.79 | −6.20 | 3.67 | 0.063 | 0.016 | |
| | subunit gamma (SEQ ID NO: 111) | | | | | | |
| P23470 | Receptor-type tyrosine-protein phosphatase | −4.79 | 1.07 | 1.14 | 0.063 | | |
| | gamma (SEQ ID NO: 27) | | | | | | |
| P12814 | Alpha-actinin-1 (SEQ ID NO: 112) | −5.56 | 1.07 | 1.14 | 0.031 | | |
| P04040 | Catalase (SEQ ID NO: 72) | −5.56 | 1.07 | 1.14 | 0.031 | | |
| P55058 | Phospholipid transfer protein (SEQ ID NO: 94) | −5.56 | −2.44 | 1.14 | 0.031 | | |
| P18669 | Phosphoglycerate mutase 1 (SEQ ID NO: 113) | −6.32 | 1.07 | 1.14 | 0.016 | | |
| P32119 | Peroxiredoxin-2 (SEQ ID NO: 77) | −6.32 | −3.94 | 1.14 | 0.016 | | |
| Q86YW5 | Trem-like transcript 1 protein (SEQ ID NO: 114) | −6.32 | 1.07 | 1.14 | 0.016 | | |
| P01233 | Choriogonadotropin subunit beta (SEQ ID | −7.09 | 1.07 | −15.58 | 0.008 | | 0.000 |
| | NO: 8) | | | | | | |
| P09211 | Glutathione S-transferase P (SEQ ID NO: 115) | −7.09 | −3.94 | 1.14 | 0.008 | | |
| Q9UIQ6 | Leucyl-cystinyl aminopeptidase (SEQ ID | −7.09 | −4.69 | −2.27 | 0.008 | 0.063 | |
| | NO: 116) | | | | | | |
| P35916 | Vascular endothelial growth factor receptor 3 | −7.86 | 1.07 | 1.14 | 0.004 | | |
| | (SEQ ID NO: 35) | | | | | | |
| Q01518 | Adenylyl cyclase-associated protein 1 (SEQ ID | −8.62 | −5.45 | 1.14 | 0.002 | 0.032 | |
| | NO: 117) | | | | | | |
| P14780 | Matrix metalloproteinase-9 (SEQ ID NO: 23) | −8.62 | −4.69 | −2.27 | 0.002 | 0.063 | |
| P62937 | Peptidyl-prolyl cis-trans isomerase A (SEQ ID | −8.62 | 1.07 | 1.14 | 0.002 | | |
| | NO: 118) | | | | | | |
| P29401 | Transketolase (SEQ ID NO: 119) | −8.62 | −4.69 | 1.14 | 0.002 | 0.063 | |
| P00558 | Phosphoglycerate kinase 1 (SEQ ID NO: 120) | −10.16 | −3.19 | 1.14 | 0.001 | | |
| P62736 | Actin, aeortic smooth muscle (SEQ ID NO: 2) | −100.83 | −88.23 | −36.59 | 0.000 | 0.000 | 0.000 |

TABLE 8
Maternal serum biomarkers to identify women with gestational hypertension (GH) or
pregnancy induced hypertension (PIH).

| | Geometric Mean Value for Each Group | | | | | | | | | |
|---|---|---|---|---|---|---|---|---|---|---|
| | Control | GH | Mild PE | Severe PE | Severe PE vs. GH | | Mild PE vs. GH | | GH vs. Control | |
| Protein | n = 58 | n = 14 | n = 30 | n = 30 | p-value | AUROC | p-value | AUROC | p-value | AUROC |
| Apolipoprotein B-100 | 24945198 | 17814400 | 18564173 | 17917397 | 0.9773 | 0.522 | 0.8211 | 0.562 | 0.0094 | 0.754 |
| Cystatin-C | 1651 | 1882 | 2191 | 2044 | 0.5049 | 0.627 | 0.0712 | 0.696 | 0.0799 | 0.641 |
| Endoglin | 55 | 59 | 87 | 108 | 0.0021 | 0.867 | 0.0152 | 0.748 | 0.4618 | 0.571 |
| Fibronectin | 228002 | 790374 | 802851 | 1297003 | 0.0135 | 0.781 | 0.9529 | 0.585 | 0.0006 | 0.901 |
| Plasma retinol-binding protein | 17573 | 19203 | 17835 | 23655 | 0.0648 | 0.714 | 0.5564 | 0.528 | 0.2954 | 0.586 |
| Apolipoprotein C-III | 92501 | 128529 | 121850 | 153721 | 0.1905 | 0.629 | 0.719 | 0.526 | 0.0145 | 0.708 |
| Chorionic somatomammotropin | 59321 | 42787 | 56699 | 29239 | 0.1892 | 0.606 | 0.1578 | 0.585 | 0.0799 | 0.665 |
| hormone | | | | | | | | | | |
| Choriogonadotropin subunit beta | 996 | 947 | 1726 | 1883 | 0.0247 | 0.728 | 0.0369 | 0.724 | 0.8573 | 0.526 |
| Pappalysin-2 | 99 | 129 | 681 | 791 | 0.0139 | 0.808 | 0.0275 | 0.767 | 0.6637 | 0.62 |
| Vascular endothelial growth factor | 60 | 42 | 65 | 40 | 0.7937 | 0.490 | 0.0865 | 0.713 | 0.1201 | 0.645 |
| receptor 3 | | | | | | | | | | |
| Histidine-rich glycoprotein | 161982 | 260038 | 184359 | 265494 | 0.9069 | 0.527 | 0.0703 | 0.664 | 0.0202 | 0.712 |
| Insulin-like growth factor-binding | 91 | 90 | 105 | 115 | 0.1382 | 0.691 | 0.1632 | 0.658 | 0.9633 | 0.525 |
| protein 2 | | | | | | | | | | |
| Matrix metalloproteinase-9 | 447 | 262 | 239 | 217 | 0.5451 | 0.532 | 0.6681 | 0.559 | 0.0155 | 0.712 |
| Pregnancy-specific beta-1- | 32534 | 29637 | 39739 | 32440 | 0.6555 | 0.592 | 0.055 | 0.675 | 0.4679 | 0.558 |
| glycoprotein 1 | | | | | | | | | | |
| C-reactive protein | 1203 | 1700 | 1070 | 1338 | 0.4683 | 0.570 | 0.1916 | 0.653 | 0.2214 | 0.629 |
| Vascular endothelial growth factor | 5 | 10 | 18 | 20 | 0.0546 | 0.805 | 0.0864 | 0.74 | 0.011 | 0.764 |
| receptor 1 | | | | | | | | | | |

Conclusions: Systematic and comprehensive maternal serum proteome analyses identified a multi-analyte panel of serum biomarkers for GH. Reliable diagnosis of GH that could distinguish from PE could facilitate early intervention strategies.

### Example 4

### Maternal Serum Biomarkers of Placental Insufficiency in Pre-Eclampsia

Objective: Preeclampsia (PE) and fetal growth restriction are associated with placental insufficiency. The early prediction of placental insufficiency associated with PE may lead to novel, early interventions to prevent fetal growth restriction. We sought to characterize maternal serum biomarkers of placental insufficiency associated with PE by proteomic analysis.

Methods: This was a secondary analysis of 57 women who developed PE from whom maternal sera was obtained between 21 and 37 weeks gestation as part of a large cohort study. None had PE at the time of sera collection. PE was defined as mild or severe following ACOG classification. Placental insufficiency was determined by umbilical artery Doppler criteria. Maternal serum proteome analysis was performed using multidimensional liquid chromatography tandem mass spectrometry (2D LC-MS/MS) and label-free quantification (spectral counting). Immunoassays were used for accurate quantification and evaluated using the Receiver Operating Characteristic (ROC) curves and logistic regression analysis.

Results: 30 patients developed mild PE and 27 developed severe PE. 13 women (12 subjects with severe PE and 1 subject with mild PE) had placental insufficiency. As shown in Table 9 below, analysis of 17 differentially expressed protein biomarkers for PE by specific immunoassay revealed 2 biomarkers with discriminant capability between those with and without placental insufficiency. PE subjects with placental insufficiency had decreased levels of chorionic somatomamotrophin1 (p-value 0.007) and pregnancy specific glycoprotein 1 (p-value 0.03) compared to women without placental insufficiency. The majority of other potential biomarkers of PE did not correlate with placental insufficiency.

TABLE 9
Role of pre-eclampsia biomarkers to distinguish PE women with and without placental
insufficiency

| | Geometric Mean Value for Each Group | | | |
|---|---|---|---|---|
| | | | PE with | Area |
| | PE with | PE without | placenta | Under |
| | placenta | placenta | insufficiency vs. | ROC |
| | insufficiency | insufficiency | without | Curve |
| Protein | n = 13 | n = 44 | p-value | (AUROC) |
| Apolipoprotein B-100 | 12119775 | 17044931 | 0.0623 | 0.646 |
| Cystatin-C | 1936 | 2160 | 0.3334 | 0.556 |
| Endoglin | 115 | 85 | 0.0512 | 0.670 |
| Fibronectin | 1524452 | 1143701 | 0.0659 | 0.669 |
| Plasma retinol-binding protein | 22099 | 20380 | 0.5171 | 0.519 |
| Apolipoprotein C-III | 129787 | 144605 | 0.4459 | 0.596 |
| Chorionic somatomammotropin | 19537 | 44991 | 0.0072 | 0.702 |
| hormone | | | | |
| Choriogonadotropin subunit beta | 2034 | 1459 | 0.2658 | 0.578 |
| Pappalysin-2 | 811 | 532 | 0.4208 | 0.514 |
| Vascular endothelial growth | 33 | 54 | 0.0646 | 0.613 |
| factor receptor 3 | | | | |
| Histidine-rich glycoprotein | 307693 | 246683 | 0.1629 | 0.660 |
| Insulin-like growth factor-binding | 123 | 102 | 0.1761 | 0.643 |
| protein 2 | | | | |
| Matrix metalloproteinase-9 | 274 | 223 | 0.3359 | 0.598 |
| Pregnancy-specific beta-1- | 25615 | 39524 | 0.0313 | 0.679 |
| glycoprotein 1 | | | | |
| C-reactive protein | 1134 | 1300 | 0.6876 | 0.518 |
| Vascular endothelial growth | 18 | 19 | 0.8587 | 0.526 |
| factor receptor 1 | | | | |

Conclusion: Placental insufficiency in PE does not correlate with biomarkers associated with the pathophysiology of active PE disease. Reliable diagnosis of placental insufficiency using maternal serum biomarkers in early gestation could facilitate new intervention strategies.

### REFERENCES

Altman, D., et al. Do women with pre-eclampsia, and their babies, benefit from magnesium sulphate? The Magpie Trial: a randomised placebo-controlled trial. Lancet, 2002. 359(9321): 1877-90.
Chappell L C, Seed P T, Briley A L, et al. Effect of antioxidants on the occurrence of preeclampsia in women at increased risk: a randomized trial. Lancet 1999; 354: 810-816.
Conde-Agudelo A, Villar J, Lindheimer M. World Health Organization systemic review of screening tests for preeclampsia. Obstet Gynecol 2004; 104: 1367-91.
Duley L, Henderson-Smart D J, Meher S, King J F. Antiplatelet agents for preventing pre-eclampsia and its complications. Cochrane Database Sys Rev. 2007; 2: CD004659.
Levine R J, Lam C, Qian C, Yu K F, Maynard S E, Sachs B P, et al. Soluble endoglin and other circulating antiangiogenic factors in preeclampsia. N Engl J Med 2006; 355: 992-1005.
Maynard, S. E., et al. Excess placental soluble fms-like tyrosine kinase 1 (sFlt1) may contribute to endothelial dysfunction, hypertension, and proteinuria in preeclampsia. J Clin Invest, 2003. 111(5): 649-58.
Polliotti B M, Fry G, Saller D N Jr., Mooney R A, Cox C, Miller R K. Second-trimester maternal serum placental growth factor and vascular endothelial growth factor for predicting severe, early-onset preeclampsia. 2003; 101: 1266-74.
Rasanen J, Tolosa J E, Girsen A, et al. Prediction of preeclampsia during early gestation using proteomic analysis of maternal serum. Am J Obstet. Gynecol. 2006; 195(S): S145
Roberts, J. M., et al., Summary of the NHLBI Working Group on Research on Hypertension During Pregnancy. Hypertension, 2003. 41(3): 437-45.
Rumbold A R, Crowther C A, Haslam R R, et al. Vitamins C and E and the risks of preeclampsia and perinatal complications. N Engl J. Med. 2006; 354 1796-806.
Rumbold A, Duley L, Crowther C, Haslam R. Antioxidants for preventing pre-eclampsia. Cochrane Database Sys Rev. 2005; 4: CD004227.
Sibai, B. M. and J. R. Barton. Expectant management of severe preeclampsia remote from term: patient selection, treatment, and delivery indications. Am J Obstet Gynecol, 2007. 196(6): 514 e1-9.
Solomon, C. G. and E. W. Seely, Hypertension in pregnancy. Endocrinol Metab Clin North Am, 2006. 35(1): 157-71, vii.
Villar J, Abdel-Aleem H, Merialdi M, et al. World Health Organization randomized trial of calcium supplementation among low calcium intake pregnant women. Am J Obstet Gynecol. 2006; 194: 639-49.
Pereira, L., A. P. Reddy, et al. (2007). “Identification of novel protein biomarkers of preterm birth in human cervical-vaginal fluid.” J Proteome Res 6(4): 1269-76.
Gravett, M. G., A. Thomas, et al. (2007). “Proteomic analysis of cervical-vaginal fluid: identification of novel biomarkers for detection of intra-amniotic infection.” J Proteome Res 6(1): 89-96.
Pang, J. X., N. Ginanni, et al. (2002). “Biomarker discovery in urine by proteomics.” J Proteome Res 1(2): 161-9.
Nagalla, S. R., J. A. Canick, et al. (2007). “Proteomic analysis of maternal serum in down syndrome: identification of novel protein biomarkers.” J Proteome Res 6(4): 1245-57.
Zybailov, B., A. L. Mosley, et al. (2006). “Statistical analysis of membrane proteome expression changes in Saccharomyces cerevisiae.” J Proteome Res 5(9): 2339-47.
Clark, M. F. and A. N. Adams (1977). “Characteristics of the microplate method of enzyme-linked immunosorbent assay for the detection of plant viruses.” J Gen Virol 34(3): 475-83.
Nerurkar, L. S., M. Namba, et al. (1984). “Rapid detection of herpes simplex virus in clinical specimens by use of a capture biotin-streptavidin enzyme-linked immunosorbent assay.” J Clin Microbiol 20(1): 109-14.
Old, W. M., K. Meyer-Arendt, et al. (2005). “Comparison of label-free methods for quantifying human proteins by shotgun proteomics.” Mol Cell Proteomics 4(10): 1487-502.
Hosmer D W and Lemeshow S. (2000) Applied Logistic Regression (2^{nd} Ed). John Wiley and Sons.
Pepe M S. (2003) The Statistical Evaluation of Medical Tests for Classification and Prediction. Oxford Univ Press.
Pepe M S. (2003) Chapter 5: Estimating the ROC Curve. The Statistical Evaluation of Medical Tests for Classification and Prediction. Oxford Univ Press.

1. A method for the diagnosis of active pre-eclampsia in a pregnant female mammalian subject comprising:
(a) testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and
(b) diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia.

2. The method of claim 1 wherein the subject is a human patient.

3. The method of claim 2, wherein said testing is implemented using an apparatus adapted to determine the level of said proteins.

4. The method of claim 2, wherein said testing is performed by using a software program executed by a suitable processor.

5. The method of claim 4, wherein the program is embodied in software stored on a tangible medium.

6. The method of claim 5 wherein the tangible medium is selected from the group consisting of a flash drive, a CD-ROM, a floppy disk, a hard drive, a DVD, and a memory associated with the processor.

7. The method of any one of claims 2 to 6, further comprising the step of preparing a report recording the results of said testing or the diagnosis.

8. The method of claim 7 wherein said report is recorded or stored on a tangible medium.

9. The method of claim 8 wherein the tangible medium is paper.

10. The method of claim 8 wherein the tangible medium is selected from the group consisting of a flash drive, a CD-ROM, a floppy disk, a hard drive, a DVD, and a memory associated with the processor.

11. The method of any one of claims 2 to 6, further comprising the step of communicating the results of said diagnosis to an interested party.

12. The method of claim 11 wherein the interested party is the patient or the attending physician.

13. The method of claim 11 wherein the communication is in writing, by email, or by telephone.

14. The method of claim 2 comprising testing the abundance of at least three of said proteins.

15. The method of claim 2 comprising testing the abundance of at least four of said proteins.

16. The method of claim 2 comprising testing the level of proteins fibronectin (Q8IVI8), choriogonadotropin subunit beta (P1233), matrix metalloproteinase-9 (P14780) and pappalysin-2 (Q9BXP8), and diagnosing said subject with pre-eclampsia, if two or more of said tested proteins shows a statistically significant difference in the maternal serum sample relative to normal maternal serum.

17. The method of claim 16 comprising diagnosing said subject with pre-eclampsia, if all of said tested proteins show a statistically significant difference in the maternal serum sample relative to normal maternal serum.

18. The method of claim 2 wherein said level is determined by an immunoassay.

19. The method of claim 2 wherein said level is determined by mass spectrometry.

20. The method of claim 2 wherein said level is determined using a protein array.

21. An immunoassay kit comprising antibodies and reagents for the detection of one or more proteins selected from the group consisting of alpha-1B-glycoprotein (P04217), actin (P62736), apolipoprotein B-100 (Q13787), apolipoprotein C-II (P02655), apolipoprotein C-III (P02656), C4b-binding protein beta chain (P20851), cathepsin D (P07339), choriogonadotropin subunit beta (P1233), cholinesterase (P06276), chorionic somatomammotropin hormone (P01243), cystatin-C (P01034), endoglin (P17813), coagulation factor XI (P03951), coagulation factor VII (P08709), fibronectin (Q8IVI8), filamin-A (P21333), heparin cofactor 2 (P05546), hepatocyte growth factor-like protein (P26927), histidine-rich glycoprotein (P04196), insulin-like growth factor-binding protein 2 (P18065), laminin subunit beta-1 (P07942), lipopolysaccharide-binding protein (P18428), matrix metalloproteinase-9 (P14780), pappalysin-2 (Q9BXP8), plastin-2 (P13796), profiling-1 (P07737), pregnancy-specific bet-1-glycoprotein (P11464), receptor-type tyrosine-protein phosphatase gamma (P23470), pregnancy zone protein (P20742), plasma retinol-binding protein (P02753), SH3 domain-binding glutamic acid-rich-like protein 3 (Q9H299), trangelin-2 (P37802), talin-1 (Q9Y490), tropomyosin alpha-4-chain (P67936), vasorin (Q6EMK4), vascular endothelial growth factor receptor 3 (P35916), vinculin (P18206), von Willebrand factor (P04275).

22. An immunoassay kit comprising antibodies and reagents for the detection of one or more proteins selected from the group consisting of cystatin-C (P01034), endoglin (P17813), fibronectin (Q8IVI8), apolipoprotein C-III (P02656), choriogonadotropin subunit beta (P1233) and pappalysin-2 (Q9BXP8).

23. The immunoassay kit of claim 22 comprising antibodies and reagents for the detection of all of said proteins.

24. An immunoassay kit comprising antibodies and reagents for the detection of one or more proteins selected from the group consisting of cystatin-C (P01034), endoglin (P17813), fibronectin (Q8IVI8), apolipoprotein C-III (P02656), and pappalysin-2 (Q9BXP8).

25. The immunoassay kit of claim 24 comprising antibodies and reagents for the detection of all of said proteins.

26. An immunoassay kit comprising antibodies and reagents for the detection of one or more proteins selected from the group consisting of fibronectin (Q8IVI8), pappalysin-2 (Q9BXP8), and matrix metalloproteinase-9 (P14780).

27. The immunoassay kit of claim 26 comprising antibodies and reagents for the detection of all of said proteins.

28. A report comprising the results of and/or diagnosis based on a test comprising
(a) testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and
(b) diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia.

29. A tangible medium storing the results of and/or diagnosis based on a test comprising
(a) testing in a maternal serum sample obtained from said subject the level of two or more proteins selected from the group consisting of apolipoprotein C-III (P02656), choriogonadotropin subunit beta (P1233), cystatin-C (P01034), endoglin (P17813), fibronectin (Q8IVI8), matrix metalloproteinase-9 (P14780), and pappalysin-2 (Q9BXP8), relative to the level in normal maternal serum or maternal serum known to be indicative of pre-eclampsia; and
(b) diagnosing said subject with pre-eclampsia if said level is determined to show a statistically significant difference relative to the level in said normal maternal serum, or is determined not to show a statistically significant difference relative to the level in said maternal serum known to be indicative of pre-eclampsia.

30. A method for the diagnosis of pre-eclampsia in a female mammalian subject in early gestation comprising:
(a) testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and
(b) diagnosing said subject with pre-eclampsia if said level shows a statistically significant difference relative to the level in said normal maternal serum, or does not show a statistically significant difference relative to the level in said maternal serum known to be indicative of pre-eclampsia.

31. The method of claim 30 wherein the subject is a human patient.

32. The method of claim 31, wherein said testing is implemented using an apparatus adapted to determine the level of said proteins.

33. The method of claim 31, wherein said testing is performed by using a software program executed by a suitable processor.

34. The method of claim 33, wherein the program is embodied in software stored on a tangible medium.

35. The method of claim 34 wherein the tangible medium is selected from the group consisting of a flash drive, a CD-ROM, a floppy disk, a hard drive, a DVD, and a memory associated with the processor.

36. The method of any one of claims 31 to 35, further comprising the step of preparing a report recording the results of said testing or the diagnosis.

37. The method of claim 36 wherein said report is recorded or stored on a tangible medium.

38. The method of claim 37 wherein the tangible medium is paper.

39. The method of claim 37 wherein the tangible medium is selected from the group consisting of a flash drive, a CD-ROM, a floppy disk, a hard drive, a DVD, and a memory associated with the processor.

40. The method of any one of claims 31 to 35, further comprising the step of communicating the results of said diagnosis to an interested party.

41. The method of claim 40 wherein the interested party is the patient or the attending physician.

42. The method of claim 40 wherein the communication is in writing, by email, or by telephone.

43. The method of claim 30 wherein the subject is about 9 to about 11 weeks gestation.

44. The method of claim 30 wherein the subject is about 10 to about 14 weeks gestation.

45. The method of claim 30 wherein the pre-eclampsia is severe pre-eclampsia.

46. The method of any one of claims 43-45 comprising testing the level of at least three of said proteins.

47. The method of any one of claims 43-45 comprising testing the level of at least four of said proteins.

48. The method of any one of claims 43-45 comprising testing the level of at least five of said proteins.

49. The method of claim 43 comprising testing the level of at least six of said proteins.

50. The method of claim 43 comprising testing the level of proteins complement factor D (P00746), vascular cell adhesion protein-1 (P19320), pappalysin-1 (Q13219), endoglin (P17813), plasma retinol-binding protein (P02753), and choriogonadotropin subunit beta (P01233).

51. The method of claim 44 comprising testing the level of proteins membrane copper amine oxidase (Q16853), C-reactive protein (P02741), Serum amyloid P-component (P02743), catalase, tubulin beta, plasma retinol binding protein, lipopolysaccharide binding protein, and chorionic somatomammotropin.

52. The method of claim 44 comprising testing the level of proteins pappalysin-1 (SEQ ID NO: 63), vascular cell adhesion protein 1 (SEQ ID NO: 60), beta-2-microglobulin (SEQ ID NO: 45), and cystatin C (SEQ ID NO: 11).

53. The method of claim 45 comprising testing the level of proteins C-reactive protein (P02741), vascular cell adhesion protein-1 (P19320), pappalysin-1 (Q13219), beta-2-microglobulin (P61769), and plasma retinol-binding protein (P02753).

54. The method of claim 30 wherein said level is determined by an immunoassay.

55. The method of claim 30 wherein said level is determined by mass spectrometry.

56. The method of claim 30 wherein said level is determined using a protein array.

57. An immunoassay kit comprising antibodies and reagents for the detection of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040).

58. An immunoassay kit comprising antibodies and reagents for the detection of two or more proteins selected from the group consisting of complement factor D (P00746), vascular cell adhesion protein-1 (P19320), and pappalysin-1 (Q13219).

59. The immunoassay kit of claim 58 comprising antibodies and reagents for the detection of all of said proteins.

60. An immunoassay kit comprising antibodies and reagents for the detection of two or more proteins selected from the group consisting of complement factor D (P00746), vascular cell adhesion protein-1 (P19320), pappalysin-1 (Q13219), endoglin (P17813), choriogonadoropin subunit beta (P01233) and plasma retinol-binding protein (P02753).

61. The immunoassay kit of claim 60 comprising antibodies and reagents for the detection of all of said proteins.

62. An immunoassay kit comprising antibodies and reagents for the detection of two or more proteins selected from the group consisting of pappalysin-1 (Q13219), C-reactive protein (P02741), plasma retinol-binding protein (P02753), beta-2-microglobulin (P61769) and vascular cell adhesion protein 1 (P19320).

63. The immunoassay kit of claim 62 comprising antibodies and reagents for the detection of all of said proteins.

64. A report comprising the results of and/or diagnosis based on a test comprising
(a) testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and
(b) diagnosing said subject with pre-eclampsia if said level shows a statistically significant difference relative to the level in said normal maternal serum, or does not show a statistically significant difference relative to the level in said maternal serum known to be indicative of pre-eclampsia.

65. A tangible medium storing the results of and/or diagnosis based on a test comprising
(a) testing in a maternal serum sample obtained from said subject the level of two or more proteins selected from the group consisting of alpha-2-antiplasmin (P08697), actin (P60709), afamin (P43652), antithrombin-III (P01008), apolipoprotein-A-II (P02652), attractin (Q9NTQ4), beta-2-microglobulin (P61769), transforming growth factor-beta-induced protein ig-h3 (Q15582), C4b-binding protein alpha chain (P04003), cathepsin D (P07339), carboxypeptidase B2 (Q961Y4), complement factor D (P00746), cartilage acidic protein 1 (Q9NQ79), dopamine beta-hydroxylase (P09172), coagulation factor XIII B chain (P05160), fibrinogen alpha chain (P02671), fibronectin (Q8IVI8), filamin A (P21333), Rho GDP-dissociation inhibitor 2 (P52566), platelet glycoprotein-1b alpha chain (P07359), haptoglobin-related protein (P00739), lipopolysaccharide-binding protein (P18418), plasma retinol-binding protein (P02753), platelet basic protein (P02775), transgelin-2 (P37802), tubulin beta-1 chain (Q9H4B7), talin-1 (Q9Y490), thymosin beta-4 (P62328), vasorin (Q6EMK4), vascular cell adhesion protein-1 (P19320), von Willebrand factor (P04275), zinc-alpha-2-glycoprotein (P25311), alpha-2-macroglobulin (P01023), apolipoprotein B-100 (Q13787), apolipoprotein C-III (P02656), choriogonadotropin subunit beta (P01233), chorionic somatomammotropin hormone (P01243), cystatin-C (P01034), endoglin (P17813), matrix metalloproteinase-9 (P14780), pappalysin-1 (Q13219), pregnancy-specific beta-1-glycoprotein 1 (Q9P1W5), vascular endothelial growth factor receptor 3 (P35916), C-reactive protein (P02741), serum amyloid P-component (P02743), membrane copper amine oxidase (Q16853), and catalase (P04040), relative to the level in normal maternal serum or maternal serum known to be indicative of pre-eclampsia; and
(b) diagnosing said subject with pre-eclampsia if said level shows a statistically significant difference relative to the level in said normal maternal serum, or does not show a statistically significant difference relative to the level in said maternal serum known to be indicative of pre-eclampsia.

66. A method for the diagnosis of gestational hypertension in a pregnant female mammalian subject comprising:
(a) testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO:115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and
(b) diagnosing said subject with gestational hypertension if said level shows a statistically significant difference relative to the level in said normal maternal serum, or does not show a statistically significant difference relative to the level in said maternal serum known to be indicative of gestational hypertension.

67. The method of claim 66 wherein the subject is a human patient.

68. The method of claim 67, wherein said testing is implemented using an apparatus adapted to determine the level of said proteins.

69. The method of claim 67, wherein said testing is performed by using a software program executed by a suitable processor.

70. The method of claim 69, wherein the program is embodied in software stored on a tangible medium.

71. The method of claim 70 wherein the tangible medium is selected from the group consisting of a flash drive, a CD-ROM, a floppy disk, a hard drive, a DVD, and a memory associated with the processor.

72. The method of any one of claims 67 to 71, further comprising the step of preparing a report recording the results of said testing or the diagnosis.

73. The method of claim 72 wherein said report is recorded or stored on a tangible medium.

74. The method of claim 73 wherein the tangible medium is paper.

75. The method of claim 73 wherein the tangible medium is selected from the group consisting of a flash drive, a CD-ROM, a floppy disk, a hard drive, a DVD, and a memory associated with the processor.

76. The method of any one of claims 67 to 71, further comprising the step of communicating the results of said diagnosis to an interested party.

77. The method of claim 76 wherein the interested party is the patient or the attending physician.

78. The method of claim 76 wherein the communication is in writing, by email, or by telephone.

79. The method of claim 67 comprising testing the level of at least three of said proteins.

80. The method of claim 67 comprising testing the level of at least four of said proteins.

81. The method of claim 67 comprising testing the level of proteins Pappalysin-2 (SEQ ID NO: 38), choriogonadotropin subunit beta (SEQ ID NO: 8), histidine rich glycoprotein (SEQ ID NO: 19), plasma retinol-binding protein (SEQ ID NO: 29), Matrix metalloproteinase-9 (SEQ ID NO: 23), Apolipoprotein B-100 (SEQ ID NO: 3), endoglin (SEQ ID NO: 12), and Vascular endothelial growth factor receptor 1 (SEQ ID NO: 121).

82. The method of claim 67 wherein said level is determined by an immunoassay.

83. The method of claim 67 wherein said level is determined by mass spectrometry.

84. The method of claim 67 wherein said level is determined using a protein array.

85. A report comprising the results of and/or diagnosis based on a test comprising
(a) testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO: 115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and
(b) diagnosing said subject with gestational hypertension if said level shows a statistically significant difference relative to the level in said normal maternal serum, or does not show a statistically significant difference relative to the level in said maternal serum known to be indicative of gestational hypertension.

86. A tangible medium storing the results of and/or diagnosis based on a test comprising
(a) testing in a maternal serum sample obtained from said subject the level of two or more proteins selected from the group consisting of cystatin-C (SEQ ID NO: 11), alpha-1-acid glycoprotein 1 (SEQ ID NO: 104), beta-2-microglobulin (SEQ ID NO: 45), cathepsin D (SEQ ID NO: 7), laminin subunit beta-1 (SEQ ID NO: 21), fibronectin (SEQ ID NO:15), chorionic somatomammotropin hormone (SEQ ID NO: 10), SH3 domain-binding glutamic acid-rich-like protein 3 (SEQ ID NO: 30), filamin-A (SEQ ID NO: 16), profilin-1 (SEQ ID NO: 25), serum amyloid P-component (SEQ ID NO: 65), fructose-biphosphate aldolase A (SEQ ID NO: 106), transgelin-2 (SEQ ID NO: 31), vinculin (SEQ ID NO: 36), cartilage acidic protein 1 (SEQ ID NO:50), plastin-2 (SEQ ID NO: 24), tropomyosin alpha-4 chain (SEQ ID NO: 33), 14-3-3 protein zeta/delta (SEQ ID NO: 108), alpha-actinin-1 (SEQ ID NO: 112), catalase (SEQ ID NO: 72), phospholipid transfer protein (SEQ ID NO: 94), phosphoglycerate mutase 1 (SEQ ID NO: 113), peroxiredoxin-2 (SEQ ID NO: 77), trem-like transcript 1 protein (SEQ ID NO: 114), choriogonadotropin subunit beta (SEQ ID NO: 8), glutathione S-transferase P (SEQ ID NO: 115), leucyl-cystinyl aminopeptidase (SEQ ID NO: 116), vascular endothelial growth factor receptor 3 (SEQ ID NO: 35), adenylyl cyclase-associated protein 1 (SEQ ID NO: 117), matrix metalloproteinase-9 (SEQ ID NO: 23), peptidyl-prolyl cis-trans isomerase A (SEQ ID NO: 118), transketolase (SEQ ID NO: 119), and phosphoglycerate kinase 1 (SEQ ID NO: 120), relative to the level in normal maternal serum or maternal serum known to be indicative of gestational hypertension; and
(b) diagnosing said subject with gestational hypertension if said level shows a statistically significant difference relative to the level in said normal maternal serum, or does not show a statistically significant difference relative to the level in said maternal serum known to be indicative of gestational hypertension.

87. A method for the diagnosis of placental insufficiency in a pregnant female mammalian subject having preeclampsia comprising:
(a) testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and
(b) diagnosing said subject with placental insufficiency if said level shows a statistically significant difference relative to the level in said normal maternal serum, or does not show a statistically significant difference relative to the level in said maternal serum known to be indicative of placental insufficiency.

88. The method of claim 87 wherein the subject is a human patient.

89. The method of claim 88, wherein said testing is implemented using an apparatus adapted to determine the level of said proteins.

90. The method of claim 88, wherein said testing is performed by using a software program executed by a suitable processor.

91. The method of claim 90, wherein the program is embodied in software stored on a tangible medium.

92. The method of claim 91 wherein the tangible medium is selected from the group consisting of a flash drive, a CD-ROM, a floppy disk, a hard drive, a DVD, and a memory associated with the processor.

93. The method of any one of claims 88 to 92, further comprising the step of preparing a report recording the results of said testing or the diagnosis.

94. The method of claim 93 wherein said report is recorded or stored on a tangible medium.

95. The method of claim 94 wherein the tangible medium is paper.

96. The method of claim 94 wherein the tangible medium is selected from the group consisting of a flash drive, a CD-ROM, a floppy disk, a hard drive, a DVD, and a memory associated with the processor.

97. The method of any one of claims 88 to 92, further comprising the step of communicating the results of said diagnosis to an interested party.

98. The method of claim 97 wherein the interested party is the patient or the attending physician.

99. The method of claim 97 wherein the communication is in writing, by email, or by telephone.

100. The method of claim 97 comprising testing the abundance of at least three of said proteins.

101. The method of claim 87 comprising testing the abundance of at least four of said proteins.

102. The method of claim 87 wherein said level is determined by an immunoassay.

103. The method of claim 87 wherein said level is determined by mass spectrometry.

104. The method of claim 87 wherein said level is determined using a protein array.

105. A report comprising the results of and/or diagnosis based on a test comprising
(a) testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and
(b) diagnosing said subject with placental insufficiency if said level shows a statistically significant difference relative to the level in said normal maternal serum, or does not show a statistically significant difference relative to the level in said maternal serum known to be indicative of placental insufficiency.

106. A tangible medium storing the results of and/or diagnosis based on a test comprising
(a) testing in a maternal serum sample obtained from said subject the level of two or more proteins selected from the group consisting of fibronectin (SEQ ID NO:15), vascular endothelial growth factor receptor 3 (SEQ ID NO: 35), chorionic somatomammortrophin (SEQ ID NO: 10), and pregnancy-specific glycoprotein (SEQ ID NO: 26), relative to the level in normal maternal serum or maternal serum known to be indicative of placental insufficiency; and
(b) diagnosing said subject with placental insufficiency if said level shows a statistically significant difference relative to the level in said normal maternal serum, or does not show a statistically significant difference relative to the level in said maternal serum known to be indicative of placental insufficiency.
